(19) United States
(12) Patent Application Publication
(10) Pub. No.: US 2025/0022596 A1
(43) Pub. Date: Jan. 16, 2025

(54) SYSTEMS AND METHODS FOR ELECTROCARDIOGRAM DEEP LEARNING INTERPRETABILITY

(71) Applicant: ICAHN SCHOOL OF MEDICINE AT MOUNT SINAI, New York, NY (US)

(72) Inventors: Hossein HONARVARGHEITANBAF, New York, NY (US); Akhil VAID, New York, NY (US); Girish NADKARNI, New York, NY (US); Benjamin GLICKSBERG, New York, NY (US)

(73) Assignee: ICAHN SCHOOL OF MEDICINE AT MOUNT SINAI, New York, NY (US)

(21) Appl. No.: 18/712,863

(22) PCT Filed: Nov. 28, 2022
(86) PCT No.: PCT/US2022/080509

§ 371 Date: May 23, 2024

Related U.S. Application Data
(60) Provisional application No. 63/283,719, filed on Nov. 29, 2021.

(51) Int. Cl. G16H 50/20

(52) U.S. Cl. CPC G16H 50/20

(57) ABSTRACT

Systems and methods for determining whether a subject has a cardiovascular abnormality are provided. An electrocardiogram of the subject is obtained, the electrocardiogram representing a plurality of time intervals summing to a total time interval and comprising, for each lead in a plurality of leads, a plurality of data points representing electronic measurements at the respective lead at a different time interval in the plurality of time intervals. For each lead, a plurality of sub-waveforms is obtained from the electrocardiogram, each sub-waveform (i) having a common duration that is less than the total time interval and that represents a fixed multiple of a reference heartbeat duration, and (ii) offset from a beginning of the electrocardiogram by a unique multiple of a sliding parameter. The plurality of sub-waveforms for each lead is inputted into a neural network comprising a plurality of parameters, obtaining an indication of cardiovascular abnormality.

### CROSS REFERENCE TO RELATED APPLICATION

This application claims priority to U.S. Provisional Patent Application No. 63/283,719 entitled “Systems and Methods for Electrocardiogram Deep Learning Interpretability,” filed Nov. 29, 2021, which is hereby incorporated by reference.

### TECHNICAL FIELD

This application is directed to systems and methods for interpretability of electrocardiograms using novel deep learning and deep learning interpretability approaches.

### BACKGROUND

Electrocardiography is a common technique for recording electrical activity of the heart over a period of time, and is used as a noninvasive, first-fline, and inexpensive diagnostic tool. This process generates electrocardiogram (ECG) data that provides physiological and structural information about the heart and is normally utilized for diagnosing cardiac-related diseases. Each ECG is obtained by placing several electrodes on the skin at different parts of the body. The arrangement of these electrodes with respect to each other is called a “lead.” Each lead records a time-dependent waveform (e.g., 10-second duration), with the magnitude being the voltage difference between the corresponding electrodes. A conventional configuration of ECGs is 12-lead with three limb leads (I, II, III), three augmented limb leads (aVF, aVL, aVR), and six precordial leads (V1, V2, V3, V4, V5, V6). The 12-lead ECG can be reduced to eight leads because leads III, aVF, aVL, aVR can be derived from the linear combination of leads I and II.

Extracting diagnostic information from ECG waveforms in an automated manner is currently feasible because of the large numbers of ECGs taken in healthcare systems. In addition, hidden patterns in the data may be present that are invisible to human experts but visible to intelligent algorithms. The field of artificial intelligence (AI) for analyzing large-scale complex data in healthcare has seen a resurgence in the past decade mainly due to large datasets, powerful computers, and new methodological developments. Therefore, it is valuable to leverage the recent advances in AI to extract important clinical information from ECGs to accelerate disease diagnosis and management.

Traditionally, statistical and machine learning (ML) methods such as support vector machines, logistic regression, random forests, and gradient boosting have been used for diagnostic predictions from ECGs. However, these methods generally require features that are manually provided by domain experts or signal processing techniques such as Fourier transform. This ad-hoc feature extraction is cumbersome and expensive in terms of time and human efforts because of the unstructured nature of ECGs, scale, and the possibility of inaccurate predictions or learning irrelevant artifacts. As part of the AI revolution, deep learning (DL) has emerged as a powerful analytics tool for large-scale unstructured data types such as text and imaging for a variety of applications in healthcare. More recently, utilizing DL for ECG data types has provided tremendous opportunities to decode complex patterns in ECG waveforms and provide valuable clinical insights.

Given the above background, what is needed in the art are systems and methods for applying deep learning techniques to electrocardiogram data to extract clinical and healthcare information.

### SUMMARY

The present disclosure addresses the problems identified in the background by providing systems and methods for electrocardiogram (ECG) deep learning (DL). As described above, the traditional use of full-duration, raw, and/or unstructured ECG waveforms in conventional ECG DL creates redundancies in feature learning and results in inaccurate predictions with large uncertainties. Accordingly, the present systems and methods provide sub-waveforms that leverage smaller units of features in ECG waveforms and thereby enhance these predictions using a data-centric, rather than a model-centric, approach.

For instance, as illustrated in Example 1 below, in a study of left ventricular dysfunction, the sub-waveforms disclosed herein not only provide improved model performance but also enhance three reliability components of explanation, interpretability, and fairness. Specifically, model performance is improved when using sub-waveforms for use as inputs to a neural network. Moreover, the clinical relevance of ECG features can be accurately interpreted and confirmed using importance scores, which further indicate that the use of sub-waveforms of ECGs result in predictions that are well aligned with clinical predictions. In addition, sub-waveforms significantly reduce prediction uncertainties within subgroups, thus improving individual fairness and relevancy of predictions.

Advantageously, by enhancing control over the granularity of ECG data used for generating predictions, the systems and methods disclosed herein provide improvements to ECG DL modeling technologies in the cardiovascular space, which can be used to improve the outcomes of patients with cardiovascular abnormalities.

Accordingly, one aspect of the present disclosure provides a computer system for determining whether a subject has a cardiovascular abnormality. The computer system comprises one or more processors and memory addressable by the one or more processors, the memory storing at least one program for execution by the one or more processors. The at least one program comprises instructions for a method of obtaining an electrocardiogram of the subject, where the electrocardiogram represents a plurality of time intervals summing to a total time interval, where each time interval in the plurality of time intervals has a first duration and the plurality of time intervals comprises at least 400 time intervals. The electrocardiogram includes, for each respective lead in a plurality of leads, a corresponding plurality of data points, each respective data point in the plurality of data points representing an electronic measurement at the respective lead at a different time interval in the plurality of time intervals.

For each respective lead in the plurality of leads, a corresponding plurality of sub-waveforms is obtained from the electrocardiogram. Each respective sub-waveform in the corresponding plurality of sub-waveforms (i) has a common duration that is less than the total time interval, where the common duration represents a fixed multiple of a reference heartbeat duration, and (ii) is offset from a beginning of the electrocardiogram by a unique multiple of a sliding parameter.

The corresponding plurality of sub-waveforms for each lead in the plurality of leads is inputted into a neural network thereby obtaining an indication as to whether the subject has the cardiovascular abnormality, where the neural network comprises a plurality of parameters (e.g., 500 or more parameters).

In some embodiments, the cardiovascular abnormality is left ventricular systolic dysfunction (LVSD) or asymptomatic left ventricular dysfunction (ALVD). In some embodiments, the cardiovascular abnormality is right ventricular dysfunction.

In some embodiments, the plurality of leads consists of I, II, V1, V2, V3, V4, V5, and V6. In some embodiments, the plurality of leads comprises I, II, V1, V2, V3, V4, V5, and V6.

In some embodiments, each sub-waveform in each corresponding plurality of sub-waveforms is in the form of a one-dimensional vector.

In some embodiments, the total time interval is X seconds, where X is a positive integer of 5 or greater, the first duration is between 1 millisecond and 5 milliseconds, the reference heartbeat duration is between 0.70 seconds and 0.78 seconds, and the fixed multiple is a scalar value between 2 and 30. In some embodiments, the total time interval is 10 seconds, the first duration is 2 milliseconds, the reference heartbeat duration is 0.74 seconds, and the fixed multiple is 2.

In some embodiments, the indication is in a binary-discrete classification form, in which a first possible value outputted by the neural network indicates the subject has the cardiovascular abnormality, and a second possible value outputted by the neural network indicates the subject does not have the cardiovascular abnormality. In some embodiments, the indication is in a scalar form indicating a likelihood or probability that the subject has the cardiovascular abnormality or a likelihood or probability that the subject does not have the cardiovascular abnormality.

In some embodiments, the neural network is a convolutional neural network having a plurality of convolutional layers followed by a plurality of residual blocks, where each residual block in the plurality of residual blocks comprises a pair of convolutional layers, followed by a fully connected layer followed by a sigmoid function.

In some embodiments, the method further comprises standardizing each corresponding plurality of data points of each respective lead in the plurality of leads to have zero-mean and unit-variance.

In some embodiments, the method further comprises using the plurality of parameters to compute a saliency map of at least a subset of the plurality of parameters. In some embodiments, the method further comprises using an occlusion approach to determine a weighting of affect on the indication for each time point in the plurality of time points for at least one lead in the plurality of leads. In some embodiments, the occlusion approach is forward pass attribution.

In some embodiments, the plurality of parameters comprises 10,000 or more parameters.

Another aspect of the present disclosure provides a method for determining whether a subject has a cardiovascular abnormality, the method comprising, at a computer system comprising a memory, obtaining an electrocardiogram of the subject, where the electrocardiogram represents a plurality of time intervals summing to a total time interval, where each time interval in the plurality of time intervals has a first duration and the plurality of time intervals comprises at least 400 time intervals. The electrocardiogram comprises, for each respective lead in a plurality of leads, a corresponding plurality of data points, each respective data point in the plurality of data points representing an electronic measurement at the respective lead at a different time interval in the plurality of time intervals.

For each respective lead in the plurality of leads, a corresponding plurality of sub-waveforms is obtained from the electrocardiogram. Each respective sub-waveform in the corresponding plurality of sub-waveforms (i) has a common duration that is less than the total time interval, where the common duration represents a fixed multiple of a reference heartbeat duration, and (ii) is offset from a beginning of the electrocardiogram by a unique multiple of a sliding parameter.

The corresponding plurality of sub-waveforms for each lead in the plurality of leads is inputted into a neural network thereby obtaining an indication as to whether the subject has the cardiovascular abnormality, where the neural network comprises a plurality of parameters (e.g., 500 or more parameters).

Another aspect of the present disclosure provides a non-transitory computer readable storage medium. The non-transitory computer readable storage medium stores instructions, which when executed by a computer system, cause the computer system to perform a method for determining whether a subject has a cardiovascular abnormality. The method comprises obtaining an electrocardiogram of the subject, where the electrocardiogram represents a plurality of time intervals summing to a total time interval, where each time interval in the plurality of time intervals has a first duration and the plurality of time intervals comprises at least 400 time intervals. The electrocardiogram comprises, for each respective lead in a plurality of leads, a corresponding plurality of data points, each respective data point in the plurality of data points representing an electronic measurement at the respective lead at a different time interval in the plurality of time intervals.

For each respective lead in the plurality of leads, a corresponding plurality of sub-waveforms is obtained from the electrocardiogram. Each respective sub-waveform in the corresponding plurality of sub-waveforms (i) has a common duration that is less than the total time interval, where the common duration represents a fixed multiple of a reference heartbeat duration, and (ii) is offset from a beginning of the electrocardiogram by a unique multiple of a sliding parameter.

The corresponding plurality of sub-waveforms for each lead in the plurality of leads is inputted into a neural network thereby obtaining an indication as to whether the subject has the cardiovascular abnormality, where the neural network comprises a plurality of parameters (e.g., 500 or more parameters).

### BRIEF DESCRIPTION OF THE DRAWINGS

In the drawings, embodiments of the systems and method of the present disclosure are illustrated by way of example. It is to be expressly understood that the description and drawings are only for the purpose of illustration and as an aid to understanding and are not intended as a definition of the limits of the systems and methods of the present disclosure.

FIGS. 1A and 1B collectively illustrate a computer system in accordance with some embodiments of the present disclosure.

FIGS. 2A, 2B, and 2C collectively illustrate methods for determining whether a subject has a cardiovascular abnormality, in which optional steps are indicated by dashed boxes, in accordance with some embodiments of the present disclosure.

FIGS. 3A, 3B, 3C, 3D, and 3E illustrate schematic views of electrocardiogram (ECG) sub-waveforms, in accordance with some embodiments of the present disclosure. FIG. 3A illustrates a full-waveform obtained directly from a 12-lead ECG device. For feeding the waveforms as input into a neural network, data points obtained from 8 independent leads (I, II, V1-V6) are used. FIG. 3B illustrates rhythmic discretization of a lead I full waveform representation 302. FIG. 3C illustrates using a sliding parameter to create offset lead I sub-waveforms from the lead I full-waveform. FIG. 3D illustrates a plurality of lead I sub-waveforms with a common duration. FIG. 3E illustrates applying the same discretization and sliding, as depicted for lead I, for the remaining 7 leads to create sub-waveforms for all 8 leads.

FIGS. 4A and 4B illustrate results obtained from systematic experiments for evaluating neural network performance of sub-waveforms with respect to full-waveform representation (as baseline), in accordance with an embodiment of the present disclosure. Performance was assessed for a left ventricular dysfunction (LVD) case study for 9,244 patients in hold-out test set. FIG. 4A illustrates the comparative performance of full-waveform (data points represented as circles) and sub-waveforms (data points represented as squares) using a minimum sliding approach, where the sliding parameter is equal to 0.74 seconds. FIG. 4B illustrates the comparative performance of full-waveform and sub-waveforms using a maximum sliding approach, where the sliding parameter is equal to the sub-waveform duration. For both experiments in FIGS. 4A and 4B, neural network performance is illustrated using both a single sub-waveform and the maximum number of sub-waveforms generated by the respective sliding approach. The optimal sub-waveform configuration is highlighted by the dashed circle in FIG. 4A and comprises 10 generated sub-waveforms, each with a duration of 1.48 seconds. The results further illustrate that the use of sub-waveforms as input to the neural network provides more accurate predictions with smaller uncertainties.

FIGS. 5A, 5B, and 5C illustrate enhanced learning by sub-waveforms for 9,244 patients in a hold-out test set, in accordance with an embodiment of the present disclosure. FIG. 5A illustrates the full-waveform heart rate for each patient (top) and heart rate histogram (bottom) for rhythmic (left panels) and arrhythmic (right panels) groups. The heart rate coefficient of variation (CV) was set to 0.01 to define the two groups. Error bars indicate averaging across different heartbeats of full-waveform. FIG. 5B illustrates a left alignment of full-waveforms (left panel) and sub-waveforms (right panel) for a plurality of 88 lead I waveforms from the rhythmic group, where the 88 lead I waveforms correspond to the heart rate with the maximum count. FIG. 5C illustrates averaging of the aligned waveforms and their saliency maps for the full-waveform (left panel) and sub-waveforms (right panel). The absolute values of a saliency map were min-max normalized for each respective waveform. As predicted by higher averaged maximum importance and lower CV, features were more aligned with less uncertainty in the sub-waveforms compared to the full-waveform representation.

FIGS. 6A, 6B, 6C and 6D illustrate an interpretation of ECG features for full-waveform representation and sub-waveforms, in accordance with an embodiment of the present disclosure. FIG. 6A illustrates a schematic of an ECG with annotated ECG features including a P-wave, PR segment, QRS complex, ST segment, and T-wave. The bounding boxes show the extents of each feature. FIG. 6B illustrates predicted probabilities obtained from a neural network for each patient in a plurality of 9,244 patients, and the corresponding confusion matrix for the full-waveform representation (left panel) and sub-waveforms (right panel). FIG. 6C illustrates positive saliency maps for a selected top five patients with positive outcome that were classified as false negative using full-waveform inputs but classified as true positive using sub-waveform inputs. The top five patients are ranked from I to V in accordance with the difference in sub-waveform and full-waveform probabilities. FIG. 6D provides, for each patient in the top five patients, a bar chart illustrating the importance scores calculated for the P, PR, QRS, ST, and T ECG features, for full-waveform representations and sub-waveforms.

FIG. 7 illustrates the impact of ECG waveform representation on 14 subgroups in a hold-out test set, in accordance with an embodiment of the present disclosure. The top plot shows prevalence of arrhythmia in each subgroup. AUROC and AUPRC for full-waveform representation (green/asterisk) and sub-waveforms (orange/no asterisk) are shown in the middle and bottom plots. The number of patients in each subgroup is shown in the parenthesis. A lower prevalence of arrhythmia in a subgroup may induce redundancies due to higher number of rhythmic full-waveforms that cause higher deep learning prediction uncertainties. The sub-waveforms provide individual fairness by reducing these disparities within a subgroup.

FIGS. 8A, 8B, 8C, 8D and 8E illustrate annotated full-waveform representations across all heartbeats and leads for each of the selected top five patients (FIG. 8A: Patient I;

FIG. 8B: Patient II; FIG. 8C: Patient III; FIG. 8D: Patient IV; FIG. 8E: Patient V) identified in FIG. 6C, in accordance with an embodiment of the present disclosure.

FIG. 9 provides an example algorithm for transforming a full-waveform to sub-waveforms, in accordance with some embodiments of the present disclosure.

FIGS. 10A and 10B collectively provide an example algorithm for computing an aligned average of a plurality of waveforms and corresponding saliency maps for a respective lead, in accordance with some embodiments of the present disclosure.

FIG. 11 provides an example algorithm for computing ECG importance scores for a waveform, for a respective lead, in accordance with some embodiments of the present disclosure.

FIGS. 12A and 12B illustrate comparative performance of a full-waveform representation and an optimal sub-waveform as inputs to a shallower neural network (FIG. 12A) and a deeper neural network (FIG. 12B), in accordance with some embodiments of the present disclosure. FIG. 12A illustrates that the full-waveform representation caused significant overfitting in the shallower neural network compared to relative stability of the sub-waveforms with respect to changes in the depth of the neural network.

Like reference numerals refer to corresponding parts throughout the several views of the drawings.

### DETAILED DESCRIPTION

As described above, what is needed in the art are systems and methods for applying deep learning techniques to electrocardiogram data to extract clinical and healthcare information, such as determining whether a subject has a cardiovascular abnormality.

One approach to solving these problems include changing the architecture of neural networks (NN) with which deep learning is performed, in order to improve the predictions from ECG waveforms [5]. For instance, two widely used architectures are convolutional neural networks (CNN) and recurrent neural networks (RNN) [9-11]. Other efforts include combining CNNs and RNNs to create convolutional recurrent neural networks (CRNN) to further enhance the predictions generated by deep learning [12]. Despite the important role of neural networks in processing the data and finding useful patterns, enhancements in learning can also be achieved by manipulations at the data level while keeping the neural network architectures fixed. For example, for many applications, the increase in performance from data preparation, labeling, preprocessing, and representation outweighs the effort of searching for an optimal neural network architecture. With emerging deep learning applications for various types of ECG data, the need for data-level manipulations has become increasingly important. In particular, each data type exhibits unique complexities and therefore benefit from specialized methods of processing and preparation.

Various methods have been implemented to investigate the impact of transforming ECG waveforms on predictions. One common strategy is augmenting waveforms by perturbing the waveforms locally or creating slices from the original waveform. For example, the window warping (WW) method perturbs the waveforms by dilating or squeezing a small region of the waveform [13]. Another popular augmentation technique is window slicing (WS) which creates random slices of the waveforms with the same label as the original full-waveform by sliding windows of the same size [14]. To alleviate the potential problems caused by random slicing, a concatenate and resampling method has been proposed, in which the waveform is sliced according to its Q points and the resulting slices are concatenated to reconstruct the length of the original waveform [15]. In addition to augmentation methods, the grid-like structural representation of waveforms for neural networks has been examined in various form, including, for instance, a 1-dimensional waveform with eight leads as channels and a 2-dimensional image with time represented as the width and leads represented as the height of the image [9, 10, 16]. Other methods include representing ECG waveforms as spectrograms using wavelet or Fourier transform techniques [17].

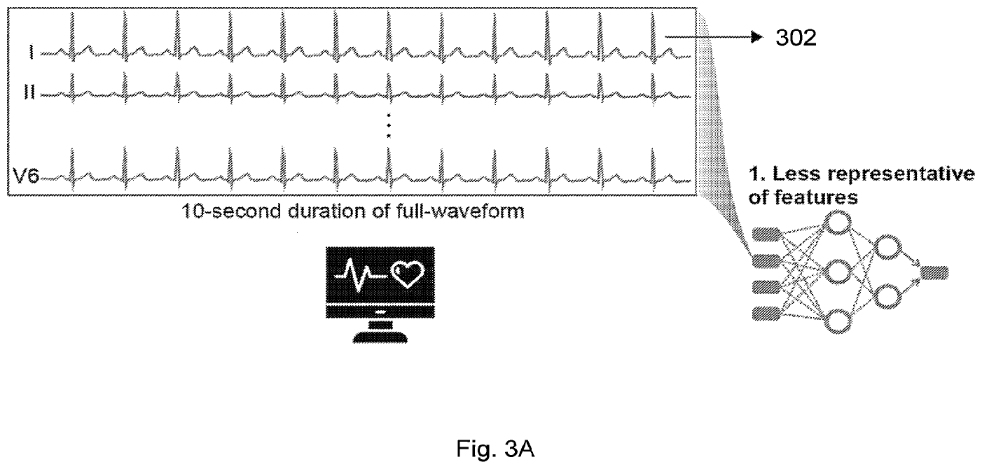

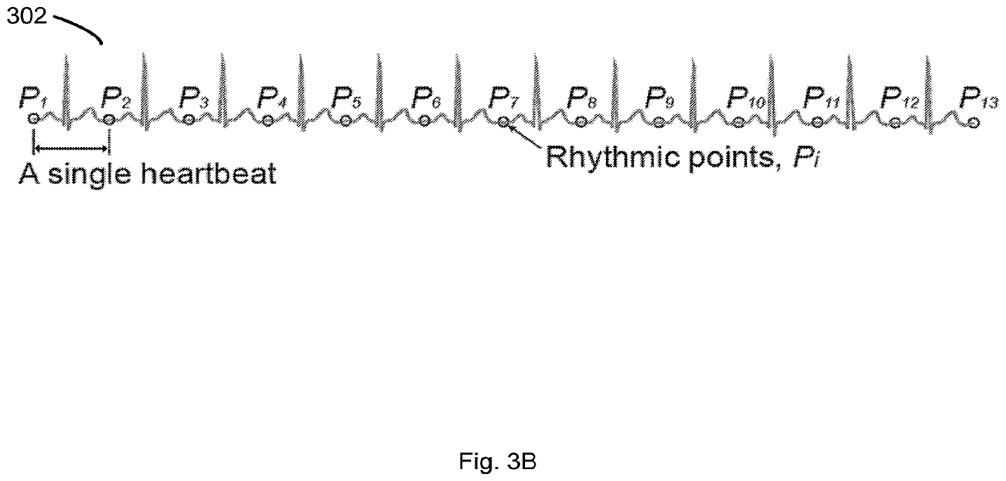

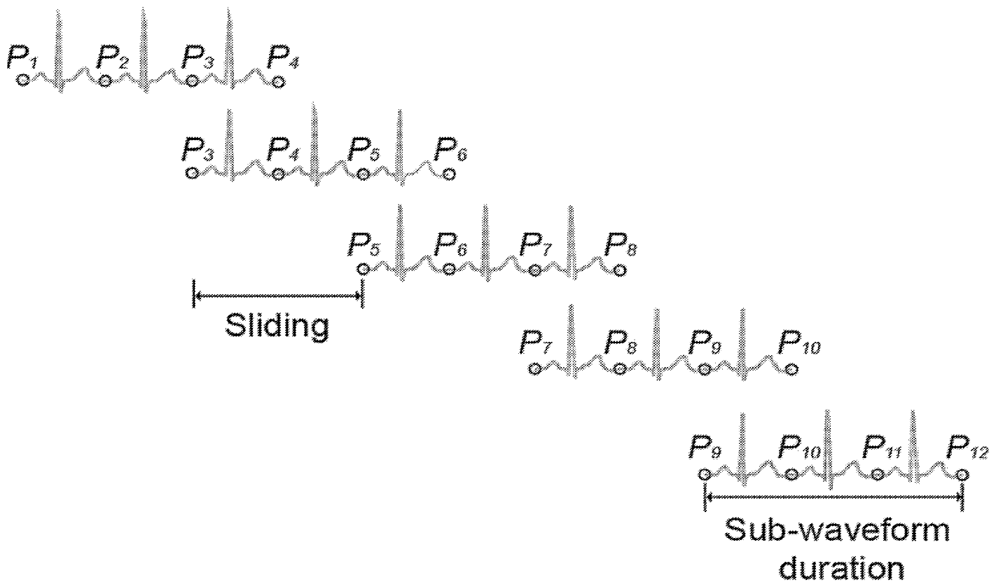

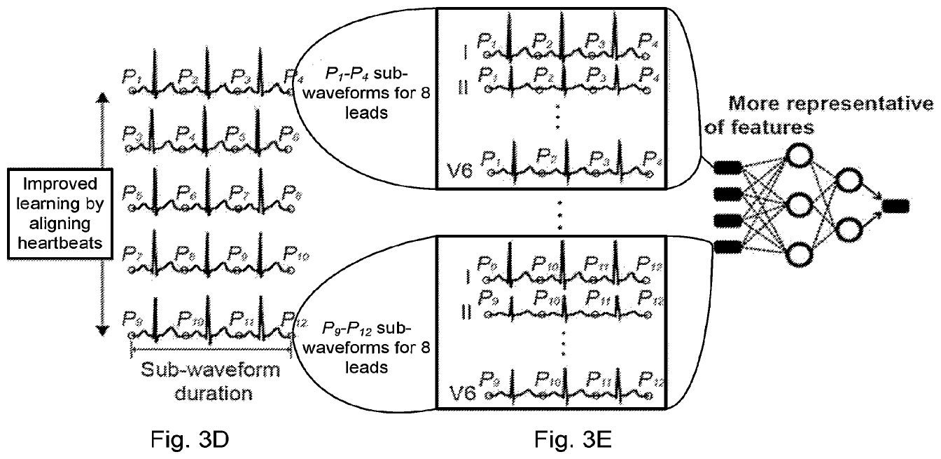

Despite such modifications, current state-of-the-art research into deep learning using ECG data nevertheless often utilizes raw waveforms obtained directly from ECG devices. These waveforms are referred to as full-waveform representations (see, e.g., FIG. 3A). As described above, the use of such unstructured ECG waveforms creates redundancies in feature learning and results in inaccurate predictions due to large uncertainties or weighting of irrelevant artifacts. Thus, optimal and reliable representations of ECG data at the waveform level for use in deep learning methodologies are needed in the art.

Accordingly, the present disclosure provides systems and methods for determining whether a subject has a cardiovascular abnormality, using sub-waveforms of ECG waveforms that improve feature learning over full-waveform representations. In particular, each sub-waveform of a respective ECG has a common duration that represents a fixed multiple of a reference heartbeat duration and is less than a total time interval (e.g., the total duration) of the corresponding full-waveform representation, and each sub-waveform is offset from the beginning of the ECG by a unique multiple of a sliding parameter. Using the plurality of sub-waveforms as inputs, an indication is obtained from a neural network indicating whether the subject has the cardiovascular abnormality.

Advantageously, the systems and method of the present disclosure utilize a data-centric approach that differs from the conventional model-centric approaches described above. In particular, the generation of sub-waveforms includes a transformation of ECG waveforms that can be performed independently from adjustments of the neural network architecture. Such transformation introduces a new space for learning optimal features and has the capacity to be used for any architecture and any task.

Moreover, in contrast to conventional augmentation approaches that utilize either the full or near-full length of the original full-waveform, the systems and methods disclosed herein reduce the number of redundant features inputted to the neural network by subdividing ECGs comprising repeating heartbeats into sub-waveforms of a common duration and using the discretized sub-waveforms as individual inputs for the neural network. This subdivision further serves, in some instances, to increase the accuracy of predictions generated by the neural network by increasing the number of training examples that are applied for each respective subject. In some implementations, the generation of sub-waveforms from ECGs comprises a rhythmic discretization that is distinct from the conventional slicing-based augmentation techniques described above, where the rhythmic discretization is based on a reference heartbeat duration (e.g., obtained from the heart rate of a reference ECG). In this way, the generation of sub-waveforms is based upon a heartbeat distribution across sub-waveforms that is approximated to be uniform, and each respective sub-waveform in the plurality of sub-waveforms for a respective ECG thus has a common structure that can be grouped and/or aligned according to its component heartbeat(s) (e.g., “heartbeat alignment”). As such, the present disclosure provides an optimal representation of ECGs for input into neural networks by creating sub-waveforms with resolution on the heartbeat-level. In an example embodiment, these sub-waveforms allow for the alignment of heartbeats for rhythmic ECGs with minimal negative impact on arrhythmic ECGs, while improving the optimality and reliability of ECG deep learning predictions.

For instance, in some embodiments, the use of sub-waveforms as inputs into neural networks provides improvements for diagnosing left ventricular dysfunction (LVD), a cardiovascular abnormality that is present in 1.4-2.2% of the population and 9% of the elderly [18]. Accurate diagnosis is useful for preventing complications such as heart failure and reducing mortality risk. Once diagnosed, treatment strategies and device implantation are normally effective. However, conventional tools for diagnosing heart failure linked to LVD include B-type natriuretic peptide (BNP) levels, which require invasive blood draws and can result in false negatives and false positives, due to artificially depressed BNP levels in obese patients and/or artificially elevated BNP levels in patients taking certain drugs like angiotensin receptor neprilysin inhibitors or ARNIs (e.g., sacubitril-valsartan) that reduce the clearance of BNP [19]. Other methods for LVD identification include quantification of the left ventricular ejection fraction (LVEF) using echocardiograms (echo) that generate ultrasound videos of the heart [20, 21]. However, the traditional method of LVEF measurement requires labor-intensive manual assessment by clinicians and can result in inaccurate predictions with large uncertainties [22, 23]. Advantageously, AI-based techniques provide a noninvasive and low-cost screening tool for LVEF quantification and LVD identification. Though automatization, deep learning can accelerate the process and improve the accuracy of predictions [24]. In addition, in some embodiments, deep learning can be used to supplement and confirm clinician assessments, for instance by predicting LVEF from ECG waveforms with echos as ground truth [25].

Examples of improvements provided by the presently disclosed systems and methods are described below in Example 1, using ECG data for a clinical randomized trial of 22,641 adults. Using sub-waveforms, a 1.6% increase in false positive predictions and a 11.7% decrease in false negative predictions was observed compared to full-waveform representations, out of 703 positive patients and 8,543 negative patients. These results illustrate that predictions obtained using sub-waveforms to substantially reduce false negative predictions while generating only a slight increase in false positives.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein the term “classification” can refer to any number(s) or other characters(s) that are associated with a particular property of an electrocardiogram or a representation thereof. For example, a “+” symbol (or the word “positive”) can signify that a respective electrocardiogram or a representation thereof is classified as having a high probability for cardiovascular abnormality. In another example, the term “classification” can refer to a probability for particular type of cardiovascular abnormality in a plurality of types of cardiovascular abnormalities. The classification can be binary (e.g., positive or negative, yes or no) or have more levels of classification (e.g., a value from 1 to 10, from 0 to 100, and/or from 0 to 1). The terms “cutoff” and “threshold” can refer to predetermined numbers used in an operation. For example, a threshold value can be a value above or below which a particular classification applies. Either of these terms can be used in either of these contexts.

As used interchangeably herein, the term “model” or “classifier” refers to a machine learning model or algorithm. In some embodiments, a classifier is an unsupervised learning algorithm. One example of an unsupervised learning algorithm is cluster analysis.

In some embodiments, a classifier is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neural networks. In some embodiments, the classifier is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of neurons (interchangeably, “nodes”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in determining cardiovascular abnormality. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for determining cardiovascular abnormality in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the classifier is a support vector machine (SVM). SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naïve Bayes algorithms. In some embodiments, the classifier is a Naive Bayes algorithm. Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a classifier is a nearest neighbor algorithm. Nearest neighbor classifiers can be memory-based and include no classifier to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the classifier is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the classifier uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model and Hidden Markov model. In some embodiments, the classifier is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the classifier is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use of a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

As used herein, the term “parameter” refers to any coefficient or, similarly, any value of an internal or external element (e.g., a weight and/or a hyperparameter) in a model (e.g., an algorithm, regressor, and/or classifier) that can affect (e.g., modify, tailor, and/or adjust) one or more inputs, outputs, and/or functions in the model. For example, in some embodiments, a parameter refers to any coefficient, weight, and/or hyperparameter that can be used to affect the behavior, learning and/or performance of a model. In some instances, a parameter is used to increase or decrease the influence of an input (e.g., a feature) to a model. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given model but can be used in any suitable model architecture for a desired performance. In some embodiments, a parameter has a fixed value. In some embodiments, a value of a parameter is manually and/or automatically adjustable. In some embodiments, a value of a parameter is modified by a validation and/or training process for a model (e.g., by error minimization and/or backpropagation methods, as described elsewhere herein).

In some embodiments, a model of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n 1×10⁷. In some embodiments n is between 10,000 and 1×10⁷, between 100,000 and 5×10⁶, or between 500,000 and 1×10⁶.

As used herein the term “untrained model” (e.g., “untrained classifier” and/or “untrained neural network”) refers to a machine learning model or algorithm such as a neural network that has not been trained on a training dataset. In some embodiments, “training a model” refers to the process of training an untrained or partially trained model. For instance, consider the case of a plurality of input features (e.g., electrocardiograms and/or a representation thereof) discussed below. The respective input features are applied as collective input to an untrained model, in conjunction with labels indicating a ground truth (e.g., an indication of presence or absence of cardiovascular abnormality) represented by the plurality of input features (hereinafter “training dataset”) to train the untrained model on electrocardiograms, including sub-waveforms, that are indicative of the presence or absence of cardiovascular abnormality, thereby obtaining a trained model. Moreover, it will be appreciated that the term “untrained model” does not exclude the possibility that transfer learning techniques are used in such training of the untrained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. That is, in non-limiting examples of transfer learning embodiments, the untrained model receives (i) input features and labels (“training dataset”) and (ii) additional data. Typically, this additional data is in the form of coefficients (e.g., regression coefficients) that were learned from another, auxiliary training dataset.

More specifically, in some embodiments, the primary training dataset is in the form of a first two-dimensional matrix, with one axis representing electrocardiograms and/or sub-waveforms, and the other axis representing some property of respective ECGs, such as data points. Application of pattern classification techniques to the auxiliary training dataset yields a second two-dimensional matrix, where one axis is the learned coefficients, and the other axis is the property of respective ECGs in the auxiliary training dataset. Matrix multiplication of the first and second matrices by their common dimension (e.g., data points) yields a third matrix of auxiliary data that can be applied, in addition to the first matrix to the untrained model. One reason it might be useful to train the untrained model using this additional information from an auxiliary training dataset is a paucity of training objects (e.g., sub-waveforms) in one or more categories in the primary training dataset. Making use of as much of the available data as possible can increase the accuracy of classifications and thus improve outputs. Thus, in the case where an auxiliary training dataset is used to train an untrained model beyond just the primary training dataset, the auxiliary training dataset is subjected to classification techniques (e.g., principal component analysis followed by logistic regression) to learn coefficients (e.g., regression coefficients) that discriminate labels (e.g., presence or absence of cardiovascular abnormality) based on the auxiliary training dataset. Such coefficients can be multiplied against a first instance of the primary training dataset and inputted into the untrained model in conjunction with the target training dataset as collective input, in conjunction with the target training dataset. As one of skill in the art will appreciate, such transfer learning can be applied with or without any form of dimension reduction technique on the auxiliary training dataset or the primary training dataset. For instance, the auxiliary training dataset (from which coefficients are learned and used as input to the untrained model in addition to the primary training dataset) can be subjected to a dimension reduction technique prior to regression (or other form of label based classification) to learn the coefficients that are applied to the primary training dataset. Alternatively, no dimension reduction other than regression or some other form of pattern classification is used in some embodiments to learn such coefficients from the auxiliary training dataset prior to applying the coefficients to an instance of the primary training dataset (e.g., through matrix multiplication where one matrix is the coefficients learned from the auxiliary training dataset and the second matrix is an instance of the primary training dataset). Moreover, in some embodiments, rather than applying the coefficients learned from the auxiliary training dataset to the primary training dataset, such coefficients are applied (e.g., by matrix multiplication based on a common axis of data points) to the data points that were used as a basis for forming the primary training set as disclosed herein.

Moreover, while a description of a single auxiliary training dataset has been disclosed, it will be appreciated that there is no limit on the number of auxiliary training datasets that may be used to complement the primary (e.g., target) training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a model such as regression to the first auxiliary training dataset) may be applied to the second auxiliary training dataset using transfer learning techniques (e.g., the above described two-dimensional matrix multiplication), which in turn may result in a trained intermediate model whose coefficients are then applied to the primary training dataset and this, in conjunction with the primary training dataset itself, is applied to the untrained classifier. Alternatively, a first set of coefficients learned from the first auxiliary training dataset (by application of a model such as regression to the first auxiliary training dataset) and a second set of coefficients learned from the second auxiliary training dataset (by application of a model such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In either example, knowledge regarding indications of cardiovascular abnormality derived from the first and second auxiliary training datasets is used, in conjunction with the cardiovascular abnormality-labeled primary training dataset), to train the untrained model.

As used herein, the term “vector” is an enumerated list of elements, such as an array of elements, where each element has an assigned meaning. As such, the term “vector” as used in the present disclosure is interchangeable with the term “tensor.” For ease of presentation, in some instances a vector may be described as being one-dimensional. However, the present disclosure is not so limited. A vector of any dimension may be used in the present disclosure provided that a description of what each element in the vector represents is defined (e.g., that element 1 represents a data point in a plurality of data points for a sub-waveform, etc.).

### EXEMPLARY SYSTEM EMBODIMENTS

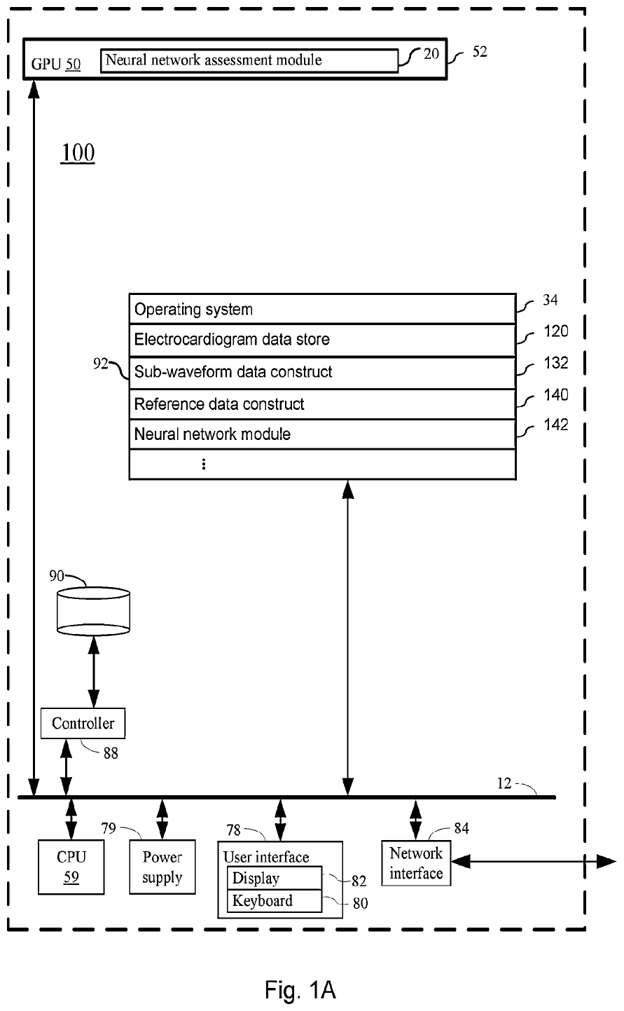

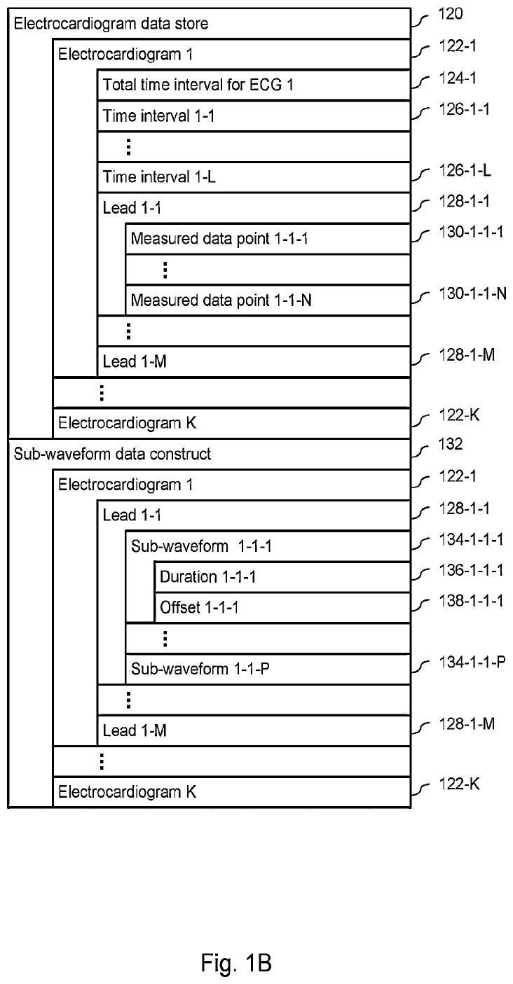

FIGS. 1A and 1B collectively illustrate a computer system 100 for determining whether a subject has a cardiovascular abnormality. For instance, it can be used to determine whether a subject has a left ventricular systolic dysfunction (LVSD), asymptomatic left ventricular dysfunction (ALVD), or right ventricular dysfunction.

Referring to FIGS. 1A-B, in typical embodiments, computer system 100 comprises one or more computers. For purposes of illustration in FIGS. 1A-B, the computer system 100 is represented as a single computer that includes all of the functionality of the disclosed computer system 100. However, the disclosure is not so limited. The functionality of the computer system 100 may be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIGS. 1A-B with the foregoing in mind, the computer system 100 comprises one or more processing units (CPUs) 59, a network or other communications interface 84, a user interface 78 (e.g., including a display 82 and optional keyboard 80 or other form of input device), a memory 92 (e.g., random access memory), one or more magnetic disk storage and/or persistent devices 90 optionally accessed by one or more controllers 88, one or more communication busses 12 for interconnecting the aforementioned components, and a power supply 79 for powering the aforementioned components. Data in memory 92 can be seamlessly shared with non-volatile memory 90 using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computer system 100 but that can be electronically accessed by the computer system 100 over an Internet, intranet, or other form of network or electronic cable using network interface 84. In some embodiments, the computer system 100 makes use of a neural network assessment module 20 that is run from the memory 52 associated with one or more graphical processing units 50 in order to improve the speed and performance of the system. In some alternative embodiments, the computer system 100 makes use of a neural network that is run from memory 92 rather than memory associated with a graphical processing unit 50.

The memory 92, memory 90, and/or optionally memory 52, of the computer system 100 stores:
an optional operating system 34 that includes procedures for handling various basic system services;
an electrocardiogram data store 120 including one or more electrocardiograms 122 (e.g., 122-1, . . . 122-K) for a respective one or more subjects, each respective electrocardiogram 122 comprising:
   a plurality of time intervals 126 (e.g., 126-1-1, . . . 126-1-L) summing to a total time interval 124 (e.g., 124-1), each respective time interval having a first duration,
   for each respective lead 128 in a plurality of leads (e.g., 128-1-1, . . . 128-1-M), a corresponding plurality of data points 130 (e.g., 130-1-1-1, . . . 130-1-1-N), each respective data point representing an electronic measurement at the respective lead at a different time interval in the plurality of time intervals;
a sub-waveform data construct 132 comprising, for each respective electrocardiogram 122 in the one or more electrocardiograms, for each respective lead 128 in the plurality of leads, a corresponding plurality of sub-waveforms 134 (e.g., 134-1-1-1, . . . 134-1-1-P) from the electrocardiogram 122 (where the corresponding plurality of sub-waveforms for a given lead is collectively the sub-waveform representation of the lead), each respective sub-waveform in the corresponding plurality of sub-waveforms including:
   a common duration 136 (e.g., 136-1-1-1) that is less than the total time interval, where the common duration represents a fixed multiple of a reference heartbeat duration, and
   an offset 138 (e.g., 138-1-1-1) from a beginning of the electrocardiogram that is a unique multiple of a sliding parameter;
optionally, a reference data construct 140 that stores at least a reference heartbeat duration; and
optionally, a neural network module 142 comprising a plurality of parameters (e.g., 500 or more parameters) that takes, as input, the corresponding plurality of sub-waveforms for each lead in the plurality of leads for a respective electrocardiogram in the one or more cardiograms and provides, as output, an indication as to whether a respective subject has a cardiovascular abnormality.

In some implementations, one or more of the above identified data elements or modules of the computer system 100 are stored in one or more of the previously mentioned memory devices and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 and/or 90 (and optionally 52) optionally stores a subset of the modules and data structures identified above. Furthermore, in some embodiments the memory 92 and/or 90 (and optionally 52) stores additional modules and data structures not described above.

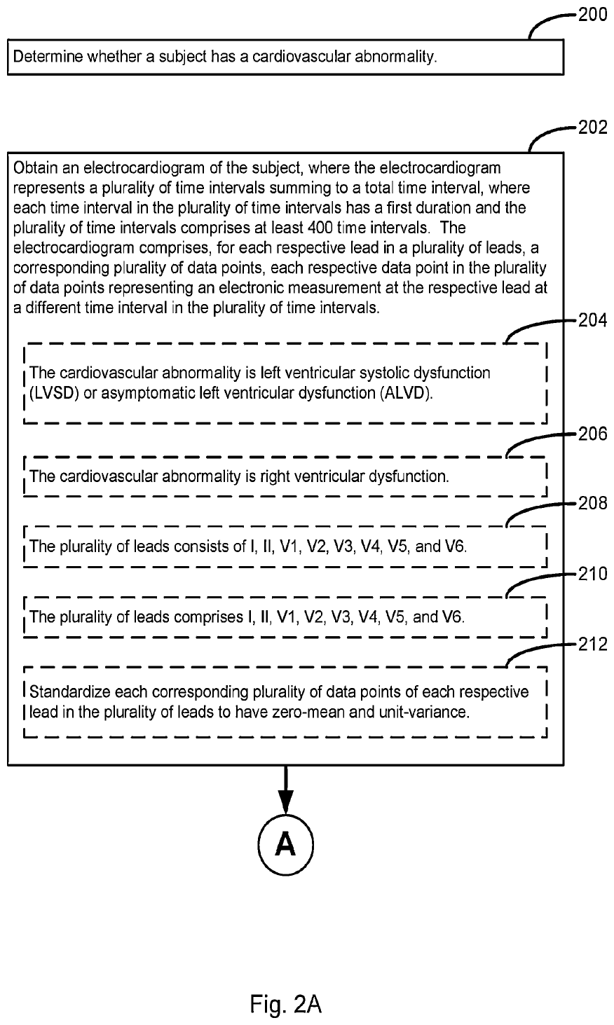

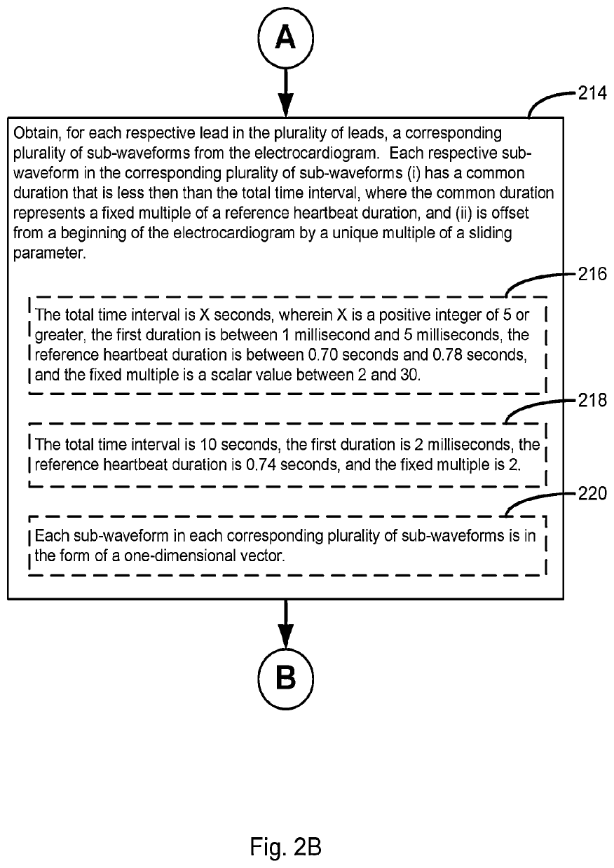

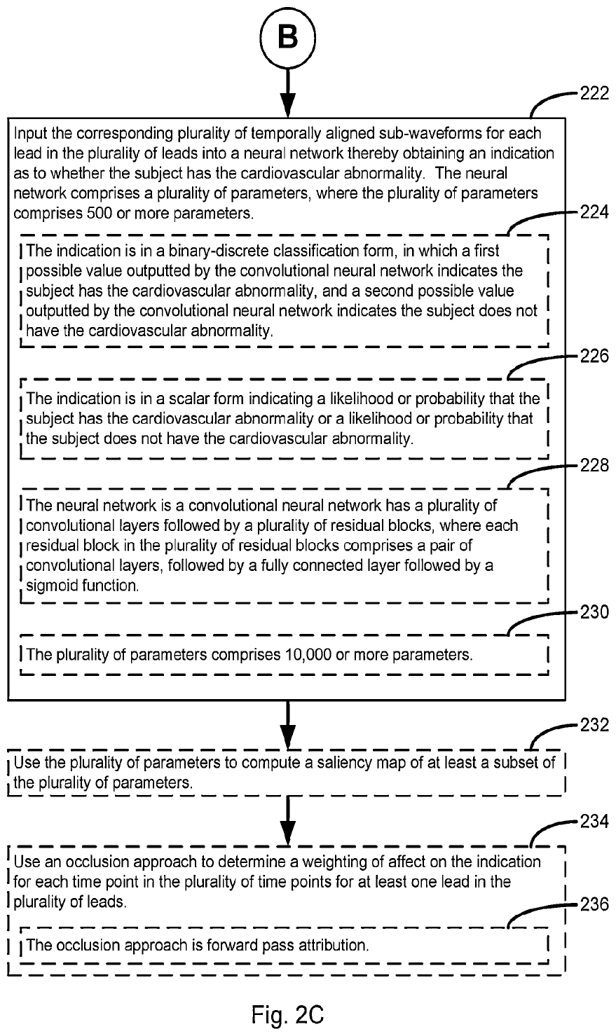

Now that a system for determining whether a subject has a cardiovascular abnormality has been disclosed, methods for performing such determination is detailed with reference to FIGS. 2A, 2B, and 2C and discussed below.

### Example Embodiments for Determining Cardiovascular Abnormality.

Referring to FIG. 2, the present disclosure provides a computer system for determining whether a subject has a cardiovascular abnormality, the computer system comprising one or more processors and memory addressable by the one or more processors. In some embodiments, the memory stores at least one program for execution by the one or more processors, the at least one program comprising instructions for a method 200.

### Electrocardiograms.

Referring to Block 202, the method 200 includes obtaining an electrocardiogram 122 of the subject, where the electrocardiogram represents a plurality of time intervals 126 summing to a total time interval 124. Each time interval in the plurality of time intervals has a first duration and the plurality of time intervals 126 comprises at least 400 time intervals. The electrocardiogram 122 comprises, for each respective lead 128 in a plurality of leads, a corresponding plurality of data points 130, each respective data point in the plurality of data points representing an electronic measurement at the respective lead at a different time interval in the plurality of time intervals.

In some embodiments, the subject is a patient. In some embodiments, the subject is obtained from a plurality of subjects. For example, in some embodiments, the method 200 comprises obtaining a respective electrocardiogram for each respective subject in a plurality of subjects, thereby obtaining a plurality of electrocardiograms. In some embodiments, the plurality of subjects is stored in a training dataset. In some embodiments, the plurality of subjects is stored in a test dataset. In some embodiments, the plurality of subjects is stored in a validation (e.g., development) dataset.

In some embodiments, the plurality of subjects comprises at least 100, at least 1000, at least 2000, at least 5000, at least 10,000, at least 20,000, at least 50,000, at least 100,000, at least 500,000, or at least 1 million subjects. In some embodiments, the plurality of subjects comprises no more than 2 million, no more than 1 million, no more than 500,000, no more than 100,000, no more than 50,000, or no more than 10,000 subjects. In some embodiments, the plurality of subjects comprises from 100 to 10,000, from 1000 to 500,000, from 100,000 to 1 million, or from 400,000 to 2 million subjects. In some embodiments, the plurality of subjects falls within another range starting no lower than 100 subjects and ending no higher than 2 million subjects.

In some embodiments, each respective subject in the plurality of subjects is associated with a single ECG. In some embodiments, one or more subjects in the plurality of subjects is associated with a respective set of ECGs.

Referring to Block 204, in some embodiments, the cardiovascular abnormality is left ventricular systolic dysfunction (LVSD) and/or asymptomatic left ventricular dysfunction (ALVD).

Left ventricular systolic dysfunction (LVSD) represents the final stage in most heart diseases. It is often asymptomatic, undetected, and untreated. In some instances, LVSD severely affects functional capacity, life-quality, and prognosis. While currently available treatments are capable of improving morbidity and mortality even in the asymptomatic, LVSD is under-diagnosed and under-treated in the elderly, especially in elderly women. Thus, early and accurate screening for LVSD is needed to improve treatment of senior citizens and to alleviate symptoms, delay progression, and improve prognosis.

Generally, clinical criteria alone are an insufficient basis for the diagnosis of LVSD, and clinical signs and symptoms alone are unreliable indicators for the detection of LVSD. For instance, these may be non-specific and obscured by co-morbidity. Echocardiography is the gold standard to diagnose LVSD and may help to determine the underlying cause of LVSD, but as access is limited, alternative screening-methods are needed. Advantageously, electrocardiogram (ECG) and NT-proBNP are inexpensive and easily obtained, and further reflect anatomy, physiology, and electro-physiology such as in the abnormal ECGs and elevated NT-proBNP with which LVSD is associated. Moreover, ECG and NT-proBNP have independent diagnostic utility, such that the combination of ECG and NT-proBNP can be used to improve screening for LVSD.

Accordingly, in some embodiments, the subject from which the respective ECG is obtained is diagnosed with a cardiovascular abnormality (e.g., left ventricular systolic dysfunction (LVSD)) based on measured left ventricular ejection fraction (LVEF).

In some embodiments, the subject is diagnosed with a cardiovascular abnormality (e.g., left ventricular systolic dysfunction (LVSD) and/or asymptomatic left ventricular dysfunction (ALVD)) when the measured left ventricular ejection fraction (LVEF) is greater than a threshold LVEF value (e.g., high LVEF). In some embodiments, the threshold LVEF value is at least 10%, at least 20%, at least 30%, at least 35%, at least 40%, or at least 50%.

In some embodiments, the subject is diagnosed as having a cardiovascular abnormality when the measured LVEF is less than a threshold LVEF value (e.g., low LVEF). In some embodiments, the threshold LVEF value is at least 10%, at least 20%, at least 30%, at least 35%, at least 40%, or at least 50%. In some embodiments, the threshold LVEF value is any number between 10% and 50% (e.g., 30%, 35%, 40%, etc.).

In some embodiments, the measured LVEF is obtained from an echo report. In some embodiments, the measured LVEF is determined using the equation: LVEF (%)=(EDV−ESV)/EDV, where EDV and ESV shows end-diastolic volume and end-systolic volume.

Additional details on using ECGs to detect LVSD are further described in, e.g., Olesen and Andersen, “ECG as a first step in the detection of left ventricular systolic dysfunction in the elderly,” ESC Heart Failure 2016; 3: 44-52; doi: 10.1002/ehf2.12067, which is hereby incorporated herein by reference in its entirety.

In some embodiments, the cardiovascular abnormality is left ventricular dysfunction (LVD). Referring to Block 206, in some embodiments, the cardiovascular abnormality is right ventricular dysfunction.

In some embodiments, the plurality of leads comprises any of three standard limb leads (Leads I, II and/or III), three augmented limb leads (aVR, aVL, and/or aVF), six chest leads (V1, V2, V3, V4, V5, and/or V6), or a combination thereof.

For instance, in some embodiments, the plurality of leads consists of 2, 3, 4, 5, 6, 7, 8, 9, 10, 11, or 12 leads. In some embodiments, the plurality of leads comprises from 4 to 12 leads, from 2 to 10 leads, or from 4 to 8 leads. In some embodiments, the plurality of leads comprises at least 2 leads, at least 4 leads, or at least 6 leads. In some embodiments, the plurality of leads comprises no more than 10 leads. For example, in some embodiments, the 12 possible leads are reduced to 8 leads because of linear dependencies of 4 leads on the other 8 leads.

See, for example, Galloway et al., “Development and Validation of a Deep-Learning Model to Screen for Hyperkalemia From the Electrocardiogram,” JAMA Cardiol. 2019; 4(5):428-436, doi:10.1001/jamacardio.2019.0640; and Cho et al., “Artificial intelligence algorithm for detecting myocardial infarction using six-lead electrocardiography,” Scientific Reports 2020; 10:20495, doi: 10.1038/s41598-020-77599-6, each of which is hereby incorporated herein by reference in its entirety.

Accordingly, referring to Block 208, in some embodiments, the plurality of leads 128 consists of I, II, V1, V2, V3, V4, V5, and V6. Referring to Block 210, in some embodiments, the plurality of leads 128 comprises I, II, V1, V2, V3, V4, V5, and V6.

In some embodiments, the electrocardiogram 122 comprises, for a single respective lead 128, a corresponding plurality of data points 130, each respective data point in the plurality of data points representing an electronic measurement at the respective lead at a different time interval in the plurality of time intervals. In some embodiments, the single respective lead is lead I.

In some embodiments, the electrocardiogram is obtained from one or more leads attached to a wearable device.

In some embodiments, the obtaining an electrocardiogram 122 of the subject comprises, for a respective lead, recording the electrocardiogram using a respective bandwidth.

In some embodiments, a respective bandwidth is at least 100 Hz, at least 200 Hz, at least 300 Hz, at least 400 Hz, at least 500 Hz, at least 600 Hz, at least 700 Hz, at least 800 Hz, or at least 1000 Hz. In some embodiments, a respective bandwidth is no more than 2000 Hz, no more than 1000 Hz, or no more than 500 Hz. In some embodiments, a respective bandwidth is from 300 to 500 Hz, from 200 to 800 Hz, or from 100 to 1000 Hz. In some embodiments, a respective bandwidth falls within another range starting no lower than 100 Hz and ending no higher than 2000 Hz.

In some embodiments, the total time interval 124 comprises at least 1, at least 2, at least 3, at least 4, at least 5, at least 10, at least 20, at least 30, or at least 60 seconds. In some embodiments, the total time interval 124 comprises at least 1, at least 2, at least 3, at least 4, at least 5, or at least 10 minutes. In some embodiments, the total time interval 124 comprises no more than 20 minutes, no more than 10 minutes, no more than 5 minutes, no more than 1 minute, or no more than 30 seconds. In some embodiments, the total time interval 124 comprises from 1 second to 30 seconds, from 5 seconds to 1 minute, from 20 seconds to 5 minutes, or from 10 seconds to 10 minutes. In some embodiments, the total time interval 124 falls within another range starting no lower than 1 second and ending no higher than 20 minutes.

In some embodiments, the plurality of time intervals 126 comprises at least 10, at least 20, at least 30, at least 50, at least 100, at least 200, at least 300, at least 400, at least 500, at least 1000, or at least 2000 time intervals. In some embodiments, the plurality of time intervals 126 comprises no more than 5000, no more than 2000, no more than 1000, no more than 500, or no more than 100 time intervals. In some embodiments, the plurality of time intervals 126 comprises from 10 to 100, from 50 to 600, from 10 to 1000, or from 200 to 2000 time intervals. In some embodiments, the plurality of time intervals 126 falls within another range starting no lower than 10 time intervals and ending no higher than 5000 time intervals.

In some embodiments, for a respective time interval 126 in the plurality of time intervals, the first duration is at least 0.1, at least 0.5, at least 1, at least 2, at least 3, at least 4, at least 5, at least 10, at least 20, at least 30, or at least 60 seconds. In some embodiments, for a respective time interval 126 in the plurality of time intervals, the first duration is no more than 5 minutes, no more than 1 minute, no more than 30 seconds, no more than 10 seconds, or no more than 1 second. In some embodiments, for a respective time interval 126 in the plurality of time intervals, the first duration is from 0.1 seconds to 1 second, from 0.5 seconds to 5 seconds, or from 1 second to 10 seconds. In some embodiments, for a respective time interval 126 in the plurality of time intervals, the first duration falls within another range starting no lower than 0.1 seconds and ending no higher than 5 minutes.

In some embodiments, for each respective time interval 126 in the plurality of time intervals, the first duration is at least 0.1, at least 0.5, at least 1, at least 2, at least 3, at least 4, at least 5, at least 10, at least 20, at least 30, or at least 60 seconds. In some embodiments, for each respective time interval 126 in the plurality of time intervals, the first duration is no more than 5 minutes, no more than 1 minute, no more than 30 seconds, no more than 10 seconds, or no more than 1 second. In some embodiments, for each respective time interval 126 in the plurality of time intervals, the first duration is from 0.1 seconds to 1 second, from 0.5 seconds to 5 seconds, or from 1 second to 10 seconds. In some embodiments, for each respective time interval 126 in the plurality of time intervals, the first duration falls within another range starting no lower than 0.1 seconds and ending no higher than 5 minutes.

In some embodiments, for a respective lead in the plurality of leads, the corresponding plurality of data points 130 comprises at least 100, at least 200, at least 500, at least 1000, at least 2000, at least 5000, at least 10,000, at least 20,000, at least 50,000, or at least 100,000 data points. In some embodiments, for a respective lead in the plurality of leads, the corresponding plurality of data points 130 comprises no more than 1 million, no more than 100,000, no more than 50,000, no more than 10,000, or no more than 1000 data points. In some embodiments, for a respective lead in the plurality of leads, the corresponding plurality of data points 130 comprises from 100 to 2000, from 1000 to 10,000, from 2000 to 8000, or from 5000 to 100,000 data points. In some embodiments, for a respective lead in the plurality of leads, the corresponding plurality of data points 130 falls within another range starting no lower than 100 data points and ending no higher than 1 million data points.

In some embodiments, for each respective lead in the plurality of leads, the corresponding plurality of data points 130 comprises at least 100, at least 200, at least 500, at least 1000, at least 2000, at least 5000, at least 10,000, at least 20,000, at least 50,000, or at least 100,000 data points. In some embodiments, for each respective lead in the plurality of leads, the corresponding plurality of data points 130 comprises no more than 1 million, no more than 100,000, no more than 50,000, no more than 10,000, or no more than 1000 data points. In some embodiments, for each respective lead in the plurality of leads, the corresponding plurality of data points 130 comprises from 100 to 2000, from 1000 to 10,000, from 2000 to 8000, or from 5000 to 100,000 data points. In some embodiments, for each respective lead in the plurality of leads, the corresponding plurality of data points 130 falls within another range starting no lower than 100 data points and ending no higher than 1 million data points.

Thus, in an example embodiment, a recording of an electrocardiogram waveform is obtained at 500 Hz for a total time interval (e.g., duration) of 10 seconds, where, for each respective lead in a plurality of 12 leads, the corresponding plurality of data points comprises at least 5000 data points.

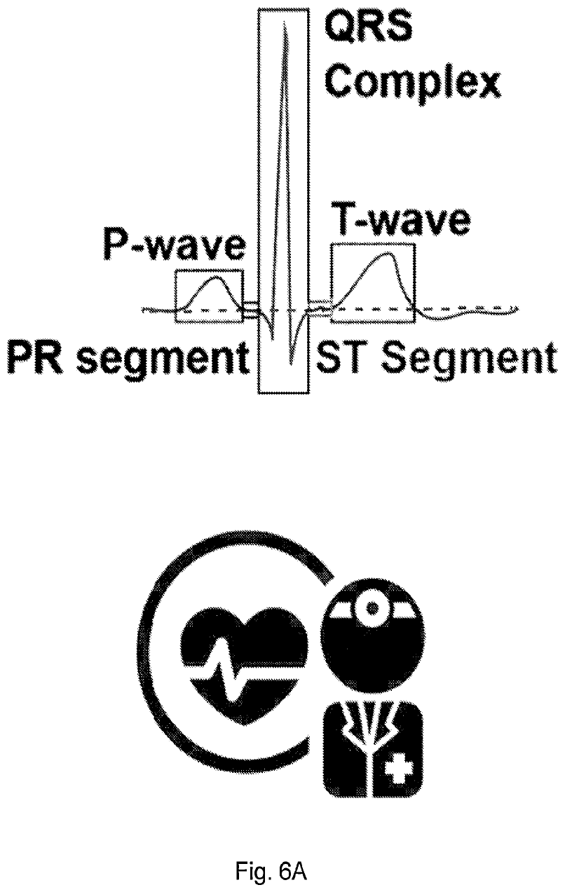

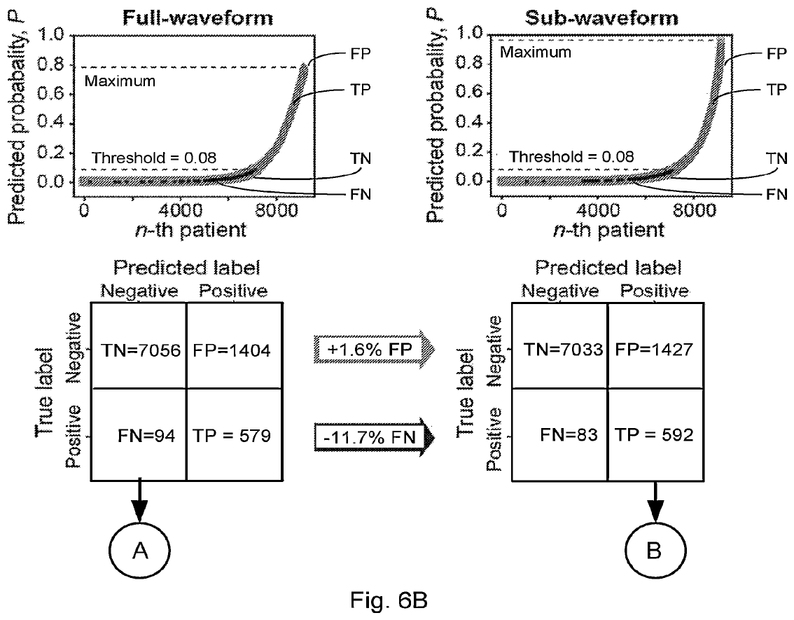

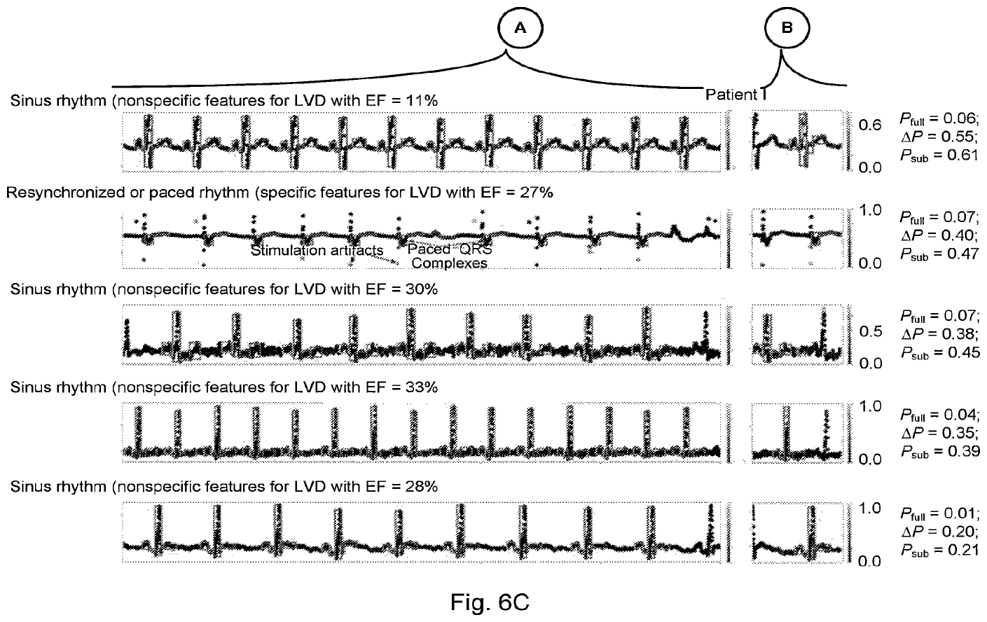

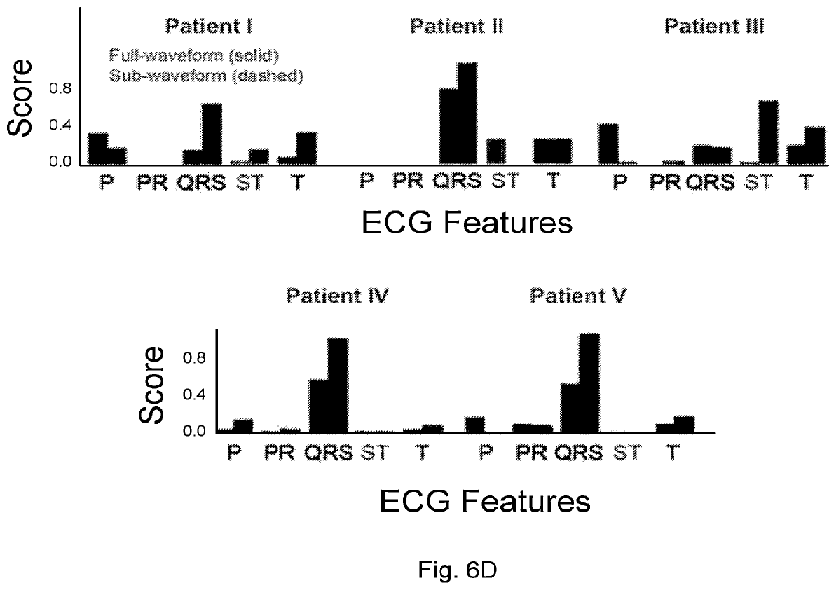

In some embodiments, the ECG comprises at least one heartbeat feature (e.g., ECG features), where the at least one heartbeat feature is one or more of a QRS complex, a P-value, a PR segment, a T-wave, and/or an ST segment. Heartbeat features are illustrated, for example, in FIG. 6A.

In some embodiments, the ECG comprises one or more heartbeats. For example, in some embodiments, the ECG comprises at least 1, at least 2, at least 3, at least 4, at least 5, at least 10, at least 15, at least 20, at least 30, at least 40, at least 50, at least 100, or at least 500 heartbeats. In some embodiments, the ECG comprises no more than 1000 no more than 500, no more than 100, no more than 50, or no more than 20 heartbeats. In some embodiments, the ECG comprises from 1 to 20, from 5 to 15, from 10 to 500, or from 8 to 50 heartbeats. In some embodiments, the ECG comprises another range of heartbeats starting no lower than 1 heartbeat and ending no higher than 1000 heartbeats.

In some embodiments, the method 200 further comprises obtaining a heart rate for the respective ECG. For example, in ECG data, heart rate is generally predicted by measuring the time between heartbeats. Thus, in some embodiments, where the ECG comprises one or more heartbeats, the obtaining the heart rate is performed by dividing the number of heartbeats in the ECG by the total time interval 124 of the ECG. In some embodiments, the ECG comprises one or more heartbeats, and the obtaining the heart rate is performed by determining a predicted heart rate for each respective heartbeat in the one or more heartbeats and averaging the plurality of predicted heart rates across the one or more heartbeats. In some embodiments, the obtaining the heart rate is performed using the corresponding plurality of data points for any one or more leads in the plurality of leads.

In some embodiments, the method 200 further comprises determining a heart rate variability based on the predicted heart rate across the one or more heartbeats in the respective ECG. For example, in some embodiments, the heart rate variability is determined by quantifying the coefficient of variation (CV) across the plurality of predicted heart rates for the respective ECG (e.g., for each respective heartbeat in the one or more heartbeats). In some embodiments, the heart rate variability is further used to classify the ECG. For example, in some embodiments, the ECG is rhythmic (CV≤0.01) or arrhythmic (CV >0.01). In some embodiments, the determining the heart rate variability is performed using the corresponding plurality of data points for any one or more leads in the plurality of leads.

Referring to rhythmic and/or arrhythmic electrocardiograms, and without being held to any one theory of operation, in some embodiments, the sub-waveforms disclosed herein can be considered to behave according to concepts in wave physics. For instance, waves in nature (e.g., light, heat, and/or electrons) can be categorized as coherent or incoherent in terms of morphology [43], where coherence refers to a wave that maintains a form of order or pattern and incoherence refers to behavior in which a wave does not follow a pattern and is naturally random or disordered. One implication of this categorization is that the coherent modes of waves provide opportunities for controlling or engineering the behavior of waves as desired. For example, this controllability can be manipulated in order to develop new technologies for the efficient control of light and heat in materials [44-46].

In some embodiments, in measuring the hemodynamics of the heart [47], ECG waveforms can be considered to behave like other physical waves that are similarly created via excitation of a given medium. Thus, in some instances, it is possible to apply concepts of wave physics to ECG waveforms. Accordingly, in some such instances, the classification of ECGs as rhythmic and arrhythmic [48] can serve as a representation of ordered (rhythmic) and disordered (arrhythmic) waveforms, which in turn resemble coherent and incoherent waves.

In some embodiments, the ECG is rhythmic and comprises a sinus rhythm.

In some embodiments, the ECG further comprises one or more metadata, including, but not limited to, patient demographics, test demographics, diagnostic information, and/or waveform details.

In some embodiments, the ECG is stored in an Extensive Markup Language (XML) file format. In some embodiments, the ECG is stored in a Hierarchical Data Format (HDF5) suitable for time series data. Any suitable file format is contemplated for use in the present disclosure, as will be apparent to one skilled in the art.

In some embodiments, the ECG is preprocessed (e.g., prior to generation of a plurality of sub-waveforms and/or prior to input to a neural network). In some embodiments, the preprocessing is performed to remove baseline drift (e.g., from baseline respiration or lead migration).

In some embodiments, the preprocessing comprises applying a filter to the ECG to remove all or a portion of the ECG that fails to satisfy a measure of central tendency threshold. In some embodiments, the measure of central tendency is any measure of central tendency known in the art, including but not limited to an arithmetic mean, weighted mean, midrange, midhinge, trimean, geometric mean, geometric median, Winsorized mean, median, and/or mode. For example, in some embodiments, the filter is a median filter that is applied to a width of all or a portion of the respective ECG. In some such embodiments, the measure of central tendency threshold is at least 0.1, at least 0.5, at least 1, or at least 5 seconds. In some embodiments, the measure of central tendency threshold is no more than 10, no more than 5, or no more than 1 seconds. In some embodiments, the measure of central tendency threshold is at least 100 Hz, at least 200 Hz, at least 500 Hz, or at least 1000 Hz. In some embodiments, the measure of central tendency threshold is no more than 2000 Hz, no more than 1000 Hz, or no more than 500 Hz. In some embodiments, the preprocessing comprises removing all or a portion of an ECG that is flagged with a warning (e.g., a poor diagnostic reading).

In some embodiments, the preprocessing comprises, for each respective lead 128 in the plurality of leads, performing a standardization for the corresponding plurality of data points 130. For example, referring to Block 212, in some embodiments, the method further comprises standardizing each corresponding plurality of data points 130 of each respective lead 128 in the plurality of leads to have zero-mean and unit-variance.

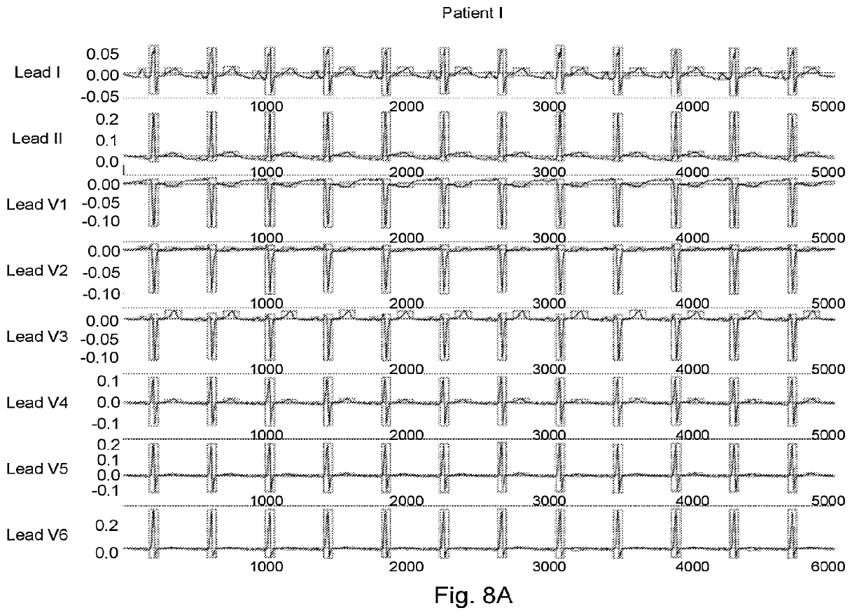

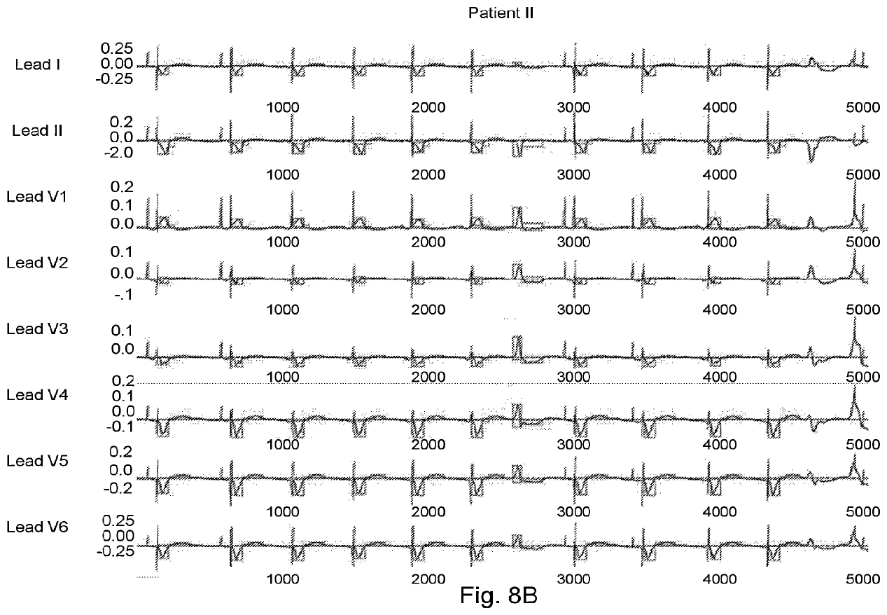

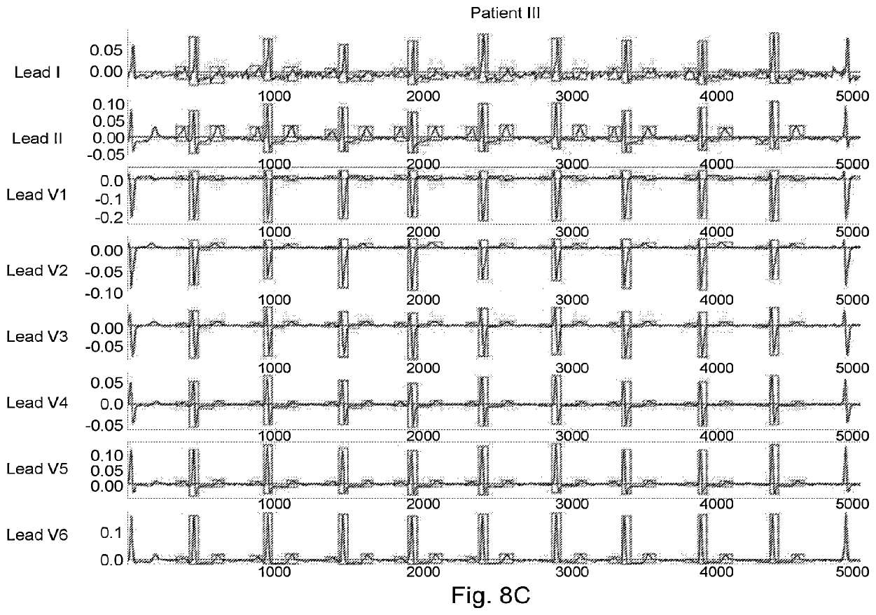

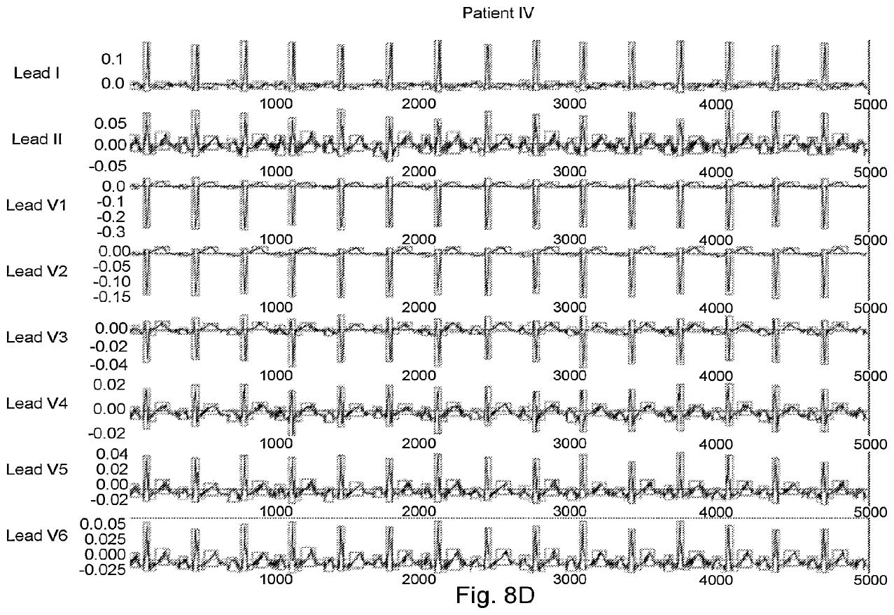

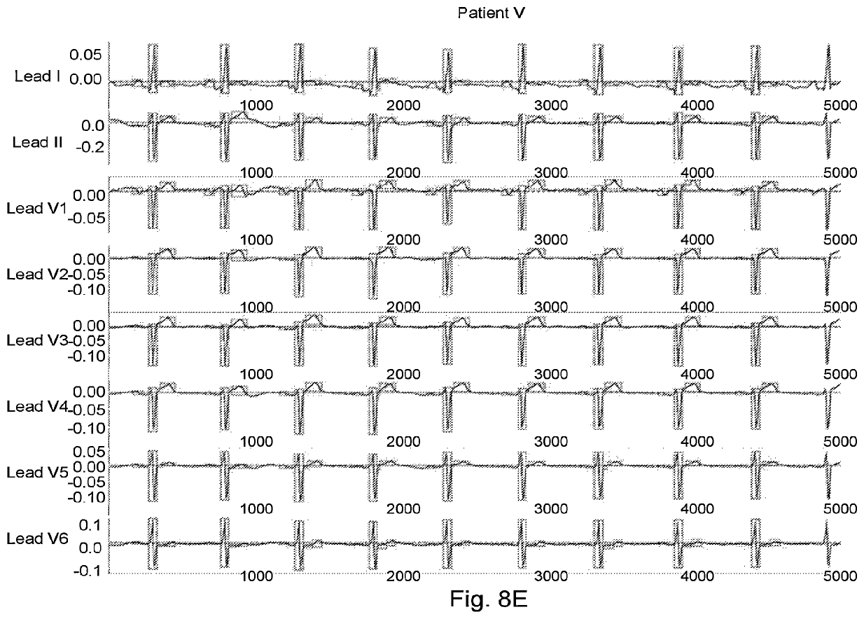

In some embodiments, the preprocessing comprises, for each respective lead 128 in the plurality of leads, annotating a respective waveform representation of the corresponding plurality of data points. For example, as illustrated in FIG. 3A, in some embodiments, the electrocardiogram comprises, for each respective lead in the plurality of leads, a visual representation of each respective data point in the corresponding plurality of data points as a waveform representation. Accordingly, in some embodiments, as illustrated in FIGS. 6A and 8A-E, the preprocessing comprises annotating each respective heartbeat in the visual representation of the ECG with at least one heartbeat feature, where the at least one heartbeat feature is one or more of a QRS complex, a P-value, a PR segment, a T-wave, and/or an ST segment.

In some embodiments, the preprocessing comprises an augmentation, including, but not limited to, window warping, window slicing, random slicing, concatenation, and/or resampling. For instance, window warping perturbs waveforms by dilating or squeezing a small region of the waveform, while window slicing creates random slices of the waveforms with the same label as the original full-waveform using sliding windows of the same size.

### Sub-Waveforms.

Referring to Block 214, the method 200 further includes obtaining, for each respective lead 128 in the plurality of leads, a corresponding plurality of sub-waveforms 134 from the electrocardiogram 122. Each respective sub-waveform in the corresponding plurality of sub-waveforms (i) has a common duration 136 that is less than the total time interval 124, where the common duration represents a fixed multiple of a reference heartbeat duration. Each respective sub-waveform in the corresponding plurality of sub-waveforms is further (ii) offset from a beginning of the electrocardiogram by a unique multiple of a sliding parameter 138.

In some embodiments, for each respective lead in the plurality of leads, each respective sub-waveform in the corresponding plurality of sub-waveforms (where the corresponding plurality of sub-waveforms is collectively considered a corresponding sub-waveform representation) comprises an ECG heartbeat feature selected from the group consisting of a QRS complex, a P-value, a PR segment, a T-wave, and/or an ST segment.

In some embodiments, for each respective lead in the plurality of leads, each respective sub-waveform in the corresponding plurality of sub-waveforms comprises one or more full heartbeats, each respective heartbeat including one or more of a QRS, P-wave, PR segment, T-wave, and ST segment. In some embodiments, each respective sub-waveform comprises all or a portion of a heartbeat.

In some embodiments, for each respective lead in the plurality of leads, the corresponding plurality of sub-waveforms from the electrocardiogram comprises at least 2, at least 3, at least 4, at least 5, at least 10, at least 15, at least 20, at least 30, at least 50, at least 100, at least 150, or at least 200 sub-waveforms. In some embodiments, for each respective lead in the plurality of leads, the corresponding plurality of sub-waveforms from the electrocardiogram comprises no more than 500, no more than 200, no more than 100, no more than 50, or no more than 20 sub-waveforms. In some embodiments, for each respective lead in the plurality of leads, the corresponding plurality of sub-waveforms from the electrocardiogram comprises from 2 to 10, from 4 to 20, from 2 to 30, from 10 to 50, or from 20 to 200 sub-waveforms. In some embodiments, for each respective lead in the plurality of leads, the corresponding plurality of sub-waveforms from the electrocardiogram falls within another range starting no lower than 2 sub-waveforms and ending no higher than 500 sub-waveforms.

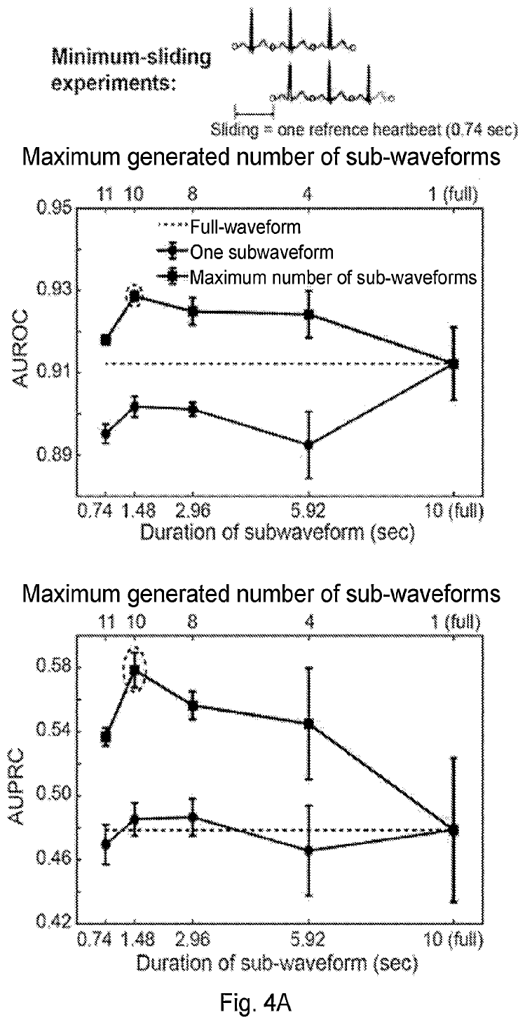

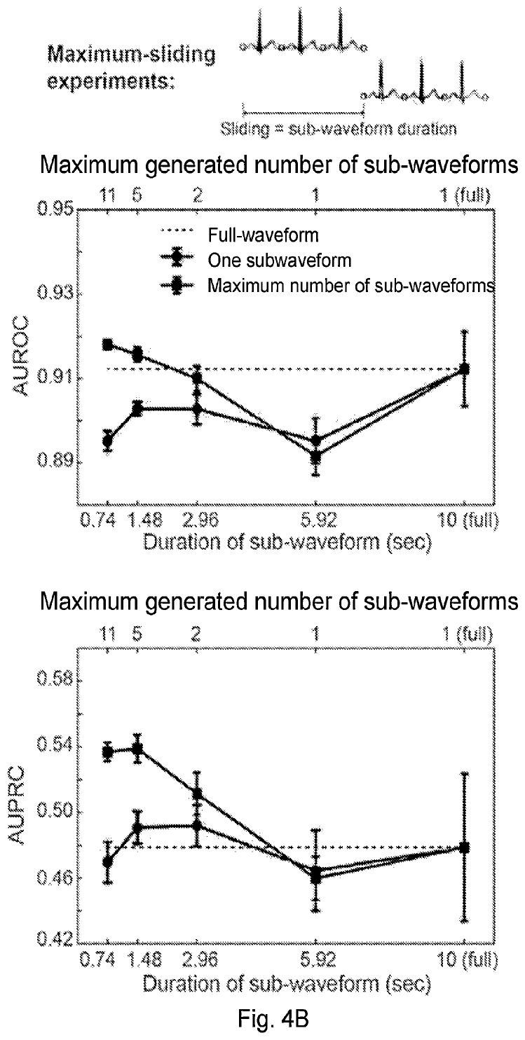

In some embodiments, the number of sub-waveforms in a corresponding plurality of sub-waveforms is determined based on a predetermined common duration 136 and an offset 138 obtained from a predetermined sliding parameter for each respective sub-waveform in the corresponding plurality of sub-waveforms. For instance, as illustrated in FIGS. 4A-B, the maximum number of generated sub-waveforms obtained from an ECG having a total time interval of 10 seconds varies depending on the predetermined common duration of the sub-waveform (e.g., duration of sub-waveform in seconds: 0.74 seconds to 10 seconds) and/or the predetermined sliding parameter of the sub-waveform (e.g., minimum-sliding by length of one reference heartbeat and/or maximum-sliding by length of sub-waveform duration). Thus, as depicted in FIG. 4A, using a predetermined sliding parameter of one reference heartbeat (e.g., 0.74 seconds) and a predetermined common duration of 1.48 seconds, the maximum generated number of sub-waveforms from a 10-second ECG is 10. Alternatively, as depicted in FIG. 4B, using a predetermined sliding parameter of the sub-waveform duration and a predetermined common duration of 1.48 seconds, the maximum generated number of sub-waveforms from a 10-second ECG is 5.

In some embodiments, the reference heartbeat duration is at least 0.3, at least 0.4, at least 0.5, at least 0.6, at least 0.7, at least 0.8, at least 0.9, at least 1, at least 1.1, at least 1.2, at least 1.3, at least 1.4, or at least 1.5 seconds. In some embodiments, the reference heartbeat duration is no more than 3, no more than 2, no more than 1, no more than 0.8, or no more than 0.5 seconds. In some embodiments, the reference heartbeat duration is from 0.3 to 1 second, from 0.5 to 1.5 seconds, from 0.6 to 0.8 seconds, or from 0.70 and 0.78 seconds. In some embodiments, the reference heartbeat duration falls within another range starting no lower than 0.3 seconds and ending no higher than 3 seconds.

In some embodiments, the reference heartbeat duration is obtained from a reference heart rate. For example, in some embodiments, the reference heart rate is from 30 to 120 bpm, from 50 to 100 bpm, from 60 to 80 bpm, or from 70 to 79 bpm.

In some embodiments, the reference heartbeat duration is a measure of central tendency (e.g., a mean, median, mode, a weighted mean, weighted median, weighted mode, etc.) of the duration of the plurality of heartbeats in a respective ECG or in a plurality of ECGs. For instance, in some embodiments, the reference heartbeat duration is obtained by averaging the duration of all the heartbeats in a respective ECG or in a plurality of ECGs. In some embodiments, the reference heart rate is determined based on a threshold percentile of a plurality of heart rates for a respective plurality of ECGs for a corresponding one or more subjects. For example, in some embodiments, the reference heart rate is selected from at least an 80^{th} percentile, at least a 90^{th} percentile, at least a 95^{th} percentile, or at least a 98^{th} percentile of a respective plurality of ECGs for a corresponding one or more subjects. In some embodiments, the obtaining the reference heartbeat duration and/or the reference heart rate is performed using the corresponding plurality of data points for any one or more leads in the plurality of leads.

In some embodiments, the fixed multiple of a reference heartbeat duration is a scalar value between 2 and 30. In some embodiments, the fixed multiple of a reference heartbeat duration is a scalar value between 1 and 100, between 2 and 10, between 3 and 6, or between 10 and 40. In some embodiments, the fixed multiple of a reference heartbeat duration is 2 or 4.

Accordingly, for each respective lead in the plurality of leads, the common duration for each respective sub-waveform in the corresponding plurality of sub-waveforms is the fixed multiple value multiplied by the duration of the reference heartbeat, described above. In some such embodiments, the common duration for each respective sub-waveform in the corresponding plurality of sub-waveforms for each respective lead in the plurality of leads is at least 0.5, at least 0.7, at least 1, at least 2, at least 3, at least 4, at least 5, at least 10, at least 20, at least 50, at least 100, or at least 150 seconds. In some embodiments, the common duration is no more than 200, no more than 100, no more than 50, no more than 20, no more than 10, or no more than 5 seconds. In some embodiments, the common duration is from 0.5 to 4 seconds, from 1 to 5 seconds, from 1 to 20 seconds, or from 5 seconds to 60 seconds. In some embodiments, the common duration falls within another range starting no lower than 0.5 seconds and ending no higher than 200 seconds.

In some embodiments, the sliding parameter is a number of heartbeats for a respective ECG.

In some embodiments, the sliding parameter is a duration of time (e.g., in seconds).

For instance, in some embodiments, the sliding parameter is a number of sub-waveforms and/or a multiple of a common duration of a sub-waveform. In some embodiments, the sliding parameter is the length of a single sub-waveform.

In some embodiments, the sliding parameter is a number of reference heartbeats and/or a multiple of a duration of a reference heartbeat.

In some embodiments, the sliding parameter is a number of reference heartbeats comprising at least 0.5, at least 1, at least 2, at least 3, at least 4, at least 5, at least 10, or at least 15 reference heartbeats. In some embodiments, the sliding parameter comprises no more than 20, no more than 10, or no more than 5 reference heartbeats. In some embodiments, the sliding parameter comprises from 0.5 to 4, from 1 to 5, from 2 to 15, or from 0.5 to 20 reference heartbeats. For instance, as illustrated in FIG. 3C, an exemplary sliding parameter comprises 2 reference heartbeats (Pᵢ).

Accordingly, in some embodiments, the unique multiple of the sliding parameter is an integer value. For example, in some such embodiments, the unique multiple of the sliding parameter is an integer value equal to n−1, for each respective sub-waveform n in a plurality of sub-waveforms, such that the respective sub-waveform n is offset from the beginning of the electrocardiogram based on the length of the sliding parameter and the number of the sliding parameter “windows” across the length of the electrocardiogram. Thus, where the sliding parameter is a number of reference heartbeats, then the unique multiple of the sliding parameter is (n−1)*the number of reference heartbeats, for each respective sub-waveform n in a plurality of sub-waveforms.

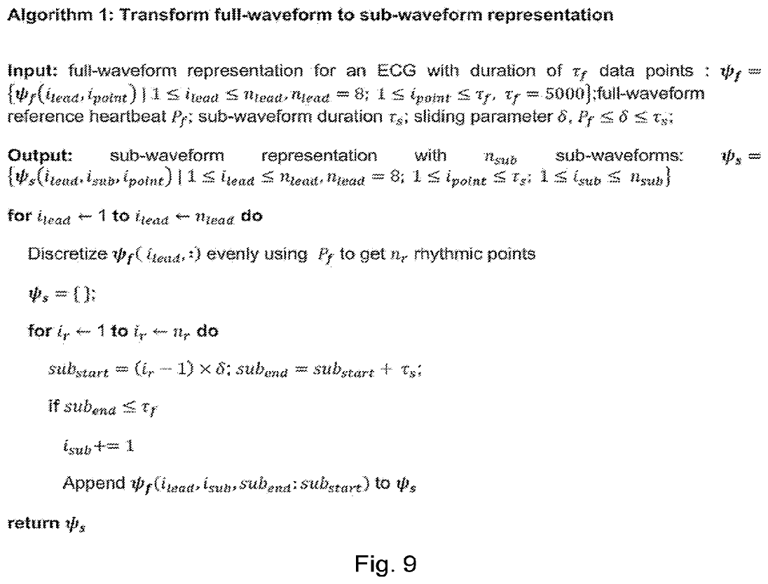

FIG. 9 provides a pseudocode for an algorithm for generating sub-waveforms from full-length ECGs, and this process is further illustrated in FIGS. 3A-E. Specifically, FIGS. 3A-E illustrate a procedure for discretizing a full-length ECG 302 for a respective lead (e.g., lead I) in a plurality of leads. The ECG 302 is subdivided into fragments (e.g., sub-waveforms) according to rhythmic points (e.g., Pᵢ for i rhythmic points). In FIG. 3B, the illustrative ECG is shown to have evenly spaced heartbeats, such that discretizing units (e.g., rhythmic points) are assigned based on the duration of discrete heartbeats; however, rhythmic points can be assigned based on an approximated discretizing unit such as a reference heartbeat. In this way, the generation of sub-waveforms can be performed even in the presence of heart rate variability across the length of the ECG.

Referring again to FIG. 3B, the rhythmic discretization based on the selected discretizing unit (e.g., a heartbeat for the respective ECG and/or a reference heartbeat) extracts sub-waveforms at rhythmic points with a duration that is a multiple of the discretizing unit (e.g., heartbeat). To diversify the sub-waveforms, the sliding parameter can be used to control the predetermined offset (e.g., where the sub-waveform should originate) and how many sub-waveforms are created depending on the predetermined common duration of the generated sub-waveforms. FIG. 3C, illustrates the process of using a sliding parameter to generate the plurality of sub-waveforms from the full-length ECG 302. For example, ECG 302 is represented as a sequence of 12 individual rhythmic points (e.g., P, for i rhythmic points, for integer i between 1 and 12). Then, consider the case where the predetermined sub-waveform common duration is 3 heartbeats, and the sliding parameter is 2 heartbeats. Thus, the first generated sub-waveform has a duration of 3 heartbeats (e.g., spanning rhythmic points P₁, P₂, P₃, P₄) and is offset from the beginning of the electrocardiogram by (n−1)*(2 heartbeats) such that the offset is zero. The second generated sub-waveform is obtained by windowing the sliding parameter once across the length of the ECG (e.g., left to right) and is offset from the beginning of the electrocardiogram by (n−1)*(2 heartbeats) such that the offset is 2 heartbeats. Accordingly, the second generated sub-waveform is characterized by the offset and the common duration of 3 heartbeats (e.g., P₃, P₄, P₅, P₆). The process is continued until the sliding parameter reaches the end of the ECG waveform and/or can no longer be further windowed across. As in FIG. 3C, for instance, using a 3-heartbeat common duration and 2-heartbeat sliding parameter results in 5 sub-waveforms that are generated from the original full-waveform.

In some embodiments, the sliding parameter continues to window across past the end of the ECG waveform such that the length of one or more sub-waveforms extend beyond the end of the ECG. In some such embodiments, the one or more sub-waveforms are padded with zero or null elements at the overhanging ends in order to retain the common duration.

FIG. 3D illustrates the generation of a plurality of the sub-waveforms from the original ECG for lead I, thus creating a new representation of the input waveform for lead I (e.g., heartbeat alignment). In some embodiments, the procedure is applied to the ECGs obtained from each respective lead in the plurality of leads, thus deriving sub-waveforms for the plurality of leads, as shown in FIG. 3E. In some such embodiments, the discretizing unit (e.g., ECG heartbeat and/or reference heartbeat), the sliding parameter, the unique multiple, and/or the common duration for creating sub-waveforms is consistent across the ECGs of each lead in the plurality of leads, thereby ensuring that the sub-waveforms used as inputs for deep learning models are the same length regardless of the respective lead from which they are obtained.

Referring to Block 216, in an example embodiment, the total time interval 124 is X seconds, where X is a positive integer of 5 or greater, the first duration is between 1 millisecond and 5 milliseconds, the reference heartbeat duration is between 0.70 seconds and 0.78 seconds, and the fixed multiple is a scalar value between 2 and 30. In another example embodiment, referring to Block 218, the total time interval 124 is 10 seconds, the first duration is 2 milliseconds, the reference heartbeat duration is 0.74 seconds, and the fixed multiple is 2.

Other configurations for sub-waveform generation are contemplated, as disclosed herein, including any substitutions, modifications, additions, deletions, and/or combinations, as will be apparent to one skilled in the art.

In some embodiments, for each respective lead in the plurality of leads, the corresponding plurality of sub-waveforms are further transformed for input to a neural network. For example, referring to Block 220, in some embodiments, each sub-waveform 134 in each corresponding plurality of sub-waveforms is in the form of a one-dimensional vector.

In some embodiments, each ECG is represented as a 1-dimensional waveform with each respective lead in the plurality of leads corresponding to a respective channel in a plurality of channels. Thus, in some embodiments, for each respective channel corresponding to a respective lead, the plurality of sub-waveforms is configured into a respective tensor such that each respective element in the respective tensor is a respective 1-dimensional vector corresponding to a respective sub-waveform.

In some embodiments, for each respective lead in the plurality of leads, the corresponding plurality of sub-waveforms are aligned prior to input to a neural network.

In some embodiments, the alignment of a respective plurality of sub-waveforms includes identifying a start location of each sub-waveform in at least one sub-waveform of the corresponding plurality of sub-waveforms and aligning each sub-waveform at the start location.

In some embodiments, the alignment of a respective plurality of sub-waveforms includes identifying a start location of a corresponding first instance of an ECG heartbeat feature, for each sub-waveform in at least one sub-waveform of the corresponding plurality of sub-waveforms, thus identifying a plurality of start locations. The alignment further includes using the plurality of start locations to temporally align each sub-waveform in each corresponding plurality of sub-waveforms, thus obtaining a corresponding plurality of temporally aligned sub-waveforms for each lead in the plurality of leads. In some such embodiments, the temporal alignment comprises aligning the start locations of the respective first instance of the ECG heartbeat features across all of the sub-waveforms.

In some embodiments, the alignment of a respective plurality of sub-waveforms includes aligning each sub-waveform in at least one sub-waveform of the corresponding plurality of sub-waveforms by a duration and/or a length of the respective sub-waveform.

In some embodiments, for each respective lead in the plurality of leads, one or more sub-waveforms in the corresponding plurality of sub-waveforms are annotated. In some embodiments, the annotation is performed using a visual representation of each respective sub-waveform in the one or more sub-waveforms. Accordingly, in some embodiments, as illustrated in FIG. 6A, the annotation comprises annotating each respective heartbeat in the visual representation of the sub-waveform with at least one heartbeat feature, where the at least one heartbeat feature is one or more of a QRS complex, a P-value, a PR segment, a T-wave, and/or an ST segment. In some embodiments, the annotation of a type of heartbeat feature for a sub-waveform of a respective ECG is consistent with a corresponding annotation of the same type of heartbeat feature that is also applied to the full-waveform representation of the respective ECG.

### Neural Networks for Determining Cardiovascular Abnormality.

Referring to Block 222, the method 200 further includes inputting the corresponding plurality of sub-waveforms 134 for each lead 128 in the plurality of leads into a neural network, thus obtaining an indication as to whether the subject has the cardiovascular abnormality, where the neural network comprises a plurality of parameters (e.g., 500 or more parameters).

In some embodiments, the indication is an output of the neural network. For example, referring to Block 224, in some embodiments, the indication is in a binary-discrete classification form, in which a first possible value outputted by the neural network indicates the subject has the cardiovascular abnormality, and a second possible value outputted by the neural network indicates the subject does not have the cardiovascular abnormality.

In some such embodiments, the indication is a discrete (e.g., binary-discrete) classification. In other words, the classification is categorical. For instance, in some embodiments, the indication is binary-discrete, and the neural network provides one value, e.g., a “1”, when the subject has the cardiovascular abnormality and another value, e.g., a “0”, when the subject does not have the cardiovascular abnormality. In some embodiments, the binary-discrete classification is 0 or 1, yes or no, true or false, positive or negative, or any other suitable binary indicator.

Referring to Block 226, in some embodiments, the indication is in a scalar form indicating a likelihood or probability that the subject has the cardiovascular abnormality or a likelihood or probability that the subject does not have the cardiovascular abnormality.

In some such embodiments, the indication is on a discrete scale that is other than binary. For instance, in some embodiments, the indication provides a first value, e.g., a “0”, when the subject has a first class of cardiovascular abnormality, a second value, e.g., a “1”, when the subject has a second class of cardiovascular abnormality, and a third value, e.g., a “2”, when the subject has a third class of cardiovascular abnormality. In some such embodiments, a respective class of cardiovascular abnormality is, e.g., a type, severity, and/or risk of cardiovascular abnormality (e.g., low, medium, and/or high risk).

Any discrete scale suitable for the indication of cardiovascular abnormality is contemplated for use in the present disclosure, including, as non-limiting examples, a discrete scale with 4, 5, 6, 7, 8, 9, 10, 11, 12, 13, 14, 15, 16, 17, 18, 19, or 20 different outcomes.

In some embodiments, the indication is on a continuous scale. The indication on the continuous scale is useful, for instance, in predicting probability that a subject has a cardiovascular abnormality. For example, in some embodiments, the indication is a value between 0 and 1, between 0 and 100, or any other suitable range of continuous values.

In some embodiments, the indication is a binary-discrete classification that is determined by a comparison of a predicted probability to a classification threshold. For example, in some such embodiments, the indication is positive or negative, where the determination that a subject is positive or negative for cardiovascular abnormality is determined based on a probability threshold (e.g., a predicted probability higher than the probability threshold is classified as positive for cardiovascular abnormality and a predicted probability that is lower than the probability threshold is classified as negative for cardiovascular abnormality).

In some embodiments, the neural network is a convolutional neural network, a deep neural network, or a combination thereof. In some embodiments, the neural network comprises any of the architectures for classifiers disclosed herein (see, Definitions: Classifiers, above) or in Hannun et al., “Cardiologist-level arrhythmia detection and classification in ambulatory electrocardiograms using a deep neural network,” Nature medicine, 2019, 25(1): p. 65-69, which is hereby incorporated herein by reference in its entirety.

Referring to Block 228, in some embodiments, the neural network is a convolutional neural network having a plurality of convolutional layers followed by a plurality of residual blocks, where each residual block in the plurality of residual blocks comprises a pair of convolutional layers, followed by a fully connected layer followed by a sigmoid function.

In some embodiments, the neural network comprises a plurality of layers (e.g., hidden layers). In some embodiments, the neural network comprises at least 3, at least 4, at least 5, at least 6, at least 7, at least 8, at least 9, at least 10, at least 20, at least 30, at least 40, at least 50, or at least 100 layers. In some embodiments, the neural network comprises no more than 200, no more than 100, no more than 50, or no more than 10 layers. In some embodiments, the neural network comprises from 1 to 10, from 1 to 20, from 2 to 80, or from 10 to 100 layers. In some embodiments, the neural network comprises a plurality of layers that falls within another range starting no lower than 3 layers and ending no higher than 100 layers.

In some embodiments, the neural network comprises a plurality of convolutional layers. In some embodiments, the neural network comprises at least 2, at least 3, at least 4, at least 5, at least 6, at least 7, at least 8, at least 9, at least 10, or at least 20 convolutional layers. In some embodiments, the neural network comprises no more than 50, no more than 20, no more than 10, or no more than 5 convolutional layers. In some embodiments, the neural network comprises from 1 to 5, from 2 to 10, from 5 to 20, or from 10 to 40 convolutional layers. In some embodiments, the neural network comprises a plurality of convolutional layers that falls within another range starting no lower than 2 convolutional layers and ending no higher than 50 convolutional layers.

In some embodiments, a convolutional layer in the plurality of convolutional layers comprises a set of learnable filters (also termed kernels). Each filter has a fixed N-dimensional size that is convolved (stepped at a predetermined step rate) across the depth, height and/or width of the input space of the convolutional layer, computing a function (e.g., a dot product) between entries (weights, or more generally parameters) of the filter and the input thereby creating a multi-dimensional activation map of that filter. In some embodiments, the filter step rate is one element, two elements, three elements, four elements, five elements, six elements, seven elements, eight elements, nine elements, ten elements, or more than ten elements of the input space.

The input space to the initial convolutional layer (e.g., output from an input layer) is formed from a vectorized representation of the input data (e.g., for each channel corresponding to a respective lead in the plurality of leads, a one-dimensional vector for each respective sub-waveform in the corresponding plurality of sub-waveforms). In some embodiments, the vectorized representation is a one-dimensional vectorized representation that serves as the input space to the initial convolutional layer.

In some embodiments, the filter is initialized (e.g., to Gaussian noise) or trained to have corresponding weights (per input channel) in which to take the mathematical operation or function of the input space values in order to compute a first single value (or set of values) of the activation layer corresponding to the filter. In some embodiment the values computed by the filter are summed, weighted, and/or biased. To compute additional values of the activation layer corresponding to the filter, the filter is then stepped (convolved) in one of the dimensions of the input space by the step rate (stride) associated with the filter, at which point the computed function or other form of mathematical operation between the filter weights and the input space values (per channel) is taken at the new location in the input space. This stepping (convolving) is repeated until the filter has sampled the entire input space in accordance with the step rate. In some embodiments, the border of the input space is zero padded to control the spatial volume of the output space produced by the convolutional layer. In typical embodiments, each of the filters of the convolutional layer canvas the entire input space in this manner thereby forming a corresponding activation map. The collection of activation maps from the filters of the convolutional layer collectively form the output space of one convolutional layer, and thereby serves as the input of a subsequent convolutional layer. Every entry in the output volume can thus also be interpreted as an output of a single neuron (or a set of neurons) that looks at a small region in the input space to the convolutional layer and shares parameters with neurons in the same activation map. Accordingly, in some embodiments, a convolutional layer in the plurality of convolutional layers has a plurality of filters and each filter in the plurality of filters convolves across the input space.

Each layer in the plurality of convolutional layers is associated with a different set of weights, or more generally a different set of parameters. With more particularity, each layer in the plurality of convolutional layers includes a plurality of filters and each filter comprises an independent plurality of parameters (e.g., weights). In some embodiments some or all such parameters (and, optionally, biases) of every filter in a given convolutional layer may be tied together, e.g., constrained to be identical.

In some embodiments, the convolutional neural network comprises an input layer that, responsive to input of a respective vector (e.g., a 1-dimensional vector representing a respective sub-waveform for a respective ECG), feeds a first plurality of values into the initial convolutional layer as a first function of values in the respective vector, where the first function is optionally computed using a graphical processing unit 50. In some embodiments, the computer system 100 has more than one graphical processing unit 50.

In some embodiments, each respective convolutional layer, other than a final convolutional layer, feeds intermediate values, as a respective second function of (i) the different set of parameters (e.g., weights) associated with the respective convolutional layer and (ii) input values received by the respective convolutional layer, into another convolutional layer in the plurality of convolutional layers. In some embodiments, the second function is computed using the graphical processing unit 50. For instance, in some embodiments, each respective filter of the respective convolutional layer canvasses the input space to the convolutional layer in accordance with the characteristic stride of the convolutional layer and at each respective filter position, takes the mathematical function of the filter parameters (e.g., weights) of the respective filter and the values of the input space at the respect filter position thereby producing a calculated point (or a set of points) on the activation layer corresponding to the respective filter position. The activation layers of the filters of the respective convolutional layer collectively represent the intermediate values of the respective convolutional layer.

In some embodiments, each respective convolutional layer in the plurality of convolutional layers further comprises a normalization step (e.g., to improve learning). In some such embodiments, the normalization step is a batch normalization. Any suitable normalization is contemplated for use in the present disclosure, as will be apparent to one skilled in the art, including but not limited to local response normalization and/or local contrast normalization, which may be applied across channels at the same position or for a particular channel across several positions. These normalization layers may encourage variety in the response of several function computations to the same input.

In some embodiments, the neural network comprises a plurality of residual blocks. In some embodiments, the neural network comprises at least 2, at least 3, at least 4, at least 5, at least 6, at least 7, at least 8, at least 9, at least 10, or at least 20 residual blocks. In some embodiments, the neural network comprises no more than 50, no more than 20, no more than 10, or no more than 5 residual blocks. In some embodiments, the neural network comprises from 1 to 5, from 2 to 10, from 5 to 20, or from 10 to 40 residual blocks. In some embodiments, the neural network comprises a plurality of residual blocks that falls within another range starting no lower than 2 residual blocks and ending no higher than 50 residual blocks. In some such embodiments, the neural network utilizes residual connections in the plurality of residual blocks to make the optimization of the model more effective by reducing the occurrence of exploding or vanishing gradients.

In some embodiments, the convolutional neural network has one or more activation layers and/or activation functions. In some embodiments, an activation layer is a layer of neurons that applies the non-saturating activation function f(x)=max(0, x). More generally, in some embodiments, a respective neuron in a plurality of neurons applies an activation function, thereby generating an output.

In some embodiments, a respective activation function increases the nonlinear properties of the decision function and of the overall network without affecting the receptive fields of the convolution layer. In other embodiments, an activation layer and/or a respective neuron includes other functions to increase nonlinearity, for example, the saturating hyperbolic tangent function f(x)=tanh, f(x)=|tanh(x)|, and the sigmoid function f(x)=(1+e^{-x})⁻¹. Nonlimiting examples of other activation functions found in some embodiments for the neural network may include, but are not limited to, logistic (or sigmoid), softmax, Gaussian, Boltzmann-weighted averaging, absolute value, linear, rectified linear, bounded rectified linear, soft rectified linear, parameterized rectified linear, average, max, min, some vector norm LP (for p=1, 2, 3, . . . , ∞), sign, square, square root, multiquadric, inverse quadratic, inverse multiquadric, polyharmonic spline, and thin plate spline.

Referring to Block 230, in some embodiments, the plurality of parameters comprises 10,000 or more parameters.

In some embodiments, the plurality of parameters comprises at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters comprises no more than 8 million, no more than 5 million, no more than 4 million, no more than 1 million, no more than 500,000, no more than 100,000, no more than 50,000, no more than 10,000, no more than 5000, or no more than 1000 parameters. In some embodiments, the plurality of parameters comprises from 100 to 5000, from 500 to 10,000, from 10,000 to 500,000, from 20,000 to 1 million, or from 1 million to 5 million parameters. In some embodiments, the plurality of parameters falls within another range starting no lower than 100 parameters and ending no higher than 8 million parameters.

One skilled in the art will appreciate that other parameters and configurations are suitable for use in the neural networks of the present disclosure, including those disclosed herein and any substitutions, modifications, additions, deletions, and/or combinations thereof.

In some embodiments, the method comprises training the neural network to determine a cardiovascular abnormality in a subject. For example, in some embodiments, the neural network is trained, responsive to receiving inputs (e.g., sub-waveforms of ECGs) corresponding to a subject, to output a prediction (e.g., probability) of whether or not the respective subject has a cardiovascular abnormality.

For instance, in some embodiments, the method comprises obtaining a plurality of sub-waveforms corresponding to a plurality of subjects, where, for each respective subject in the plurality of subjects, the corresponding plurality of sub-waveforms are labeled with ground truth indications of whether or not the subject has a cardiovascular abnormality. In some embodiments, the ground truth indications are obtained from a measurement (e.g., a measured LVEF, where the subject is diagnosed with the cardiovascular abnormality when the measured LVEF is less than a threshold LVEF value). In some embodiments, the plurality of sub-waveforms is obtained in a training dataset for a corresponding plurality of training subjects.

In some embodiments, the inputs to the neural network comprise sub-waveforms of ECGs. In some embodiments, the inputs to the neural network comprise full-waveform representations of ECGs.

Suitable embodiments for subjects (e.g., training subjects), determination and/or diagnosis of cardiovascular abnormality, obtaining ECGs, and generating sub-waveforms for the training dataset include, but are not limited to, any embodiments for subjects, diagnoses, ECGs, sub-waveforms, and methods of obtaining the same as described herein (see, e.g., the sections entitled “Electrocardiograms” and “Sub-waveforms,” above), or any substitutions, modifications, additions, deletions, and/or combinations thereof, as will be apparent to one skilled in the art.

In some embodiments, for each respective subject in the plurality of subjects represented in the training dataset, the corresponding plurality of sub-waveforms and associated ground truth label are sequentially passed through the neural network, thus generating an output indicating whether the respective subject has a cardiovascular abnormality.

In some embodiments where deep learning techniques utilize a neural network as described above, the training of the neural network to improve the accuracy of its prediction involves modifying one or more parameters, including, but not limited to, weights in the filters in the convolutional layers as well as biases in the network layers. In some embodiments, the weights and biases are further constrained with various forms of regularization such as L1, L2, weight decay, and dropout.

For instance, in some embodiments, the neural network or any of the models disclosed herein optionally, where training data is labeled (e.g., with an indication of cardiovascular abnormality), have their parameters (e.g., weights) tuned (adjusted to potentially minimize the error between the system's predicted indications and the training data's measured indications). Various methods used to minimize error function, such as gradient descent methods, include, but are not limited to, log-loss, sum of squares error, hinge-loss methods. In some embodiments, these methods further include second-order methods or approximations such as momentum, Hessian-free estimation, Nesterov's accelerated gradient, adagrad, etc. In some embodiments, the methods also combine unlabeled generative pretraining and labeled discriminative training.

Accordingly, in some embodiments, the training of the neural network comprises adjusting one or more parameters in the plurality of parameters by back-propagation through a loss function. In some embodiments, the loss function is a regression task and/or a classification task. Non-limiting examples of loss functions suitable for the regression task include, but are not limited to, a mean squared error loss function, a mean absolute error loss function, a Huber loss function, a Log-Cosh loss function, or a quantile loss function. See, Wang et al., 2020, “A Comprehensive Survey of Loss Functions in Machine Learning,” Annals of Data Science, doi.org/10.1007/s40745-020-00253-5, last accessed Sep. 15, 2021, which is hereby incorporated by reference. Non-limiting examples of loss functions suitable for the classification task include, but are not limited to, a binary cross entropy loss function, a hinge loss function, or a squared hinged loss function. In some embodiments, the loss function is any suitable regression task loss function or classification task loss function.

Other suitable methods for training the neural network that are contemplated for use in the present disclosure are further described herein (see, e.g., Definitions: Untrained model, above).

In some embodiments, the parameters of the neural network are randomly initialized prior to training.

In some embodiments, the neural network comprises a dropout regularization parameter. For example, in some embodiments, a regularization is performed by adding a penalty to the loss function, where the penalty is proportional to the values of the parameters in the trained or untrained model. Generally, regularization reduces the complexity of the model by adding a penalty to one or more parameters to decrease the importance of the respective hidden neurons associated with those parameters. Such practice can result in a more generalized model and reduce overfitting of the data. In some embodiments, the regularization includes an L1 or L2 penalty.

In some embodiments, the training the neural network comprises an optimizer. In some embodiments, the training the neural network comprises a learning rate.

In some embodiments, the learning rate is at least 0.0001, at least 0.0005, at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1. In some embodiments, the learning rate is no more than 1, no more than 0.9, no more than 0.8, no more than 0.7, no more than 0.6, no more than 0.5, no more than 0.4, no more than 0.3, no more than 0.2, no more than 0.1 no more than 0.05, no more than 0.01, or less. In some embodiments, the learning rate is from 0.0001 to 0.01, from 0.001 to 0.5, from 0.001 to 0.01, from 0.005 to 0.8, or from 0.005 to 1. In some embodiments, the learning rate falls within another range starting no lower than 0.0001 and ending no higher than 1.

In some embodiments, the learning rate further comprises a learning rate decay (e.g., a reduction in the learning rate over one or more epochs). For example, a learning decay rate can be a reduction in the learning rate of 0.5 or 0.1. In some embodiments, the learning rate is a differential learning rate. In some embodiments, the training the neural network further uses a scheduler that conditionally applies the learning rate decay based on an evaluation of a performance metric over a threshold number of training epochs (e.g., the learning rate decay is applied when the performance metric fails to satisfy a threshold performance value for at least a threshold number of training epochs).

In some embodiments, the performance of the neural network is measured at one or more time points using a performance metric, including, but not limited to, a training loss metric, a validation loss metric, and/or a mean absolute error. In some embodiments, the performance metric is an area under receiving operating characteristic (AUROC) and/or an area under precision-recall curve (AUPRC).

For instance, in some embodiments, the performance of the neural network is measured by validating the model using a validation (e.g., development) dataset. In some such embodiments, the training the neural network forms a trained neural network when the neural network satisfies a minimum performance requirement based on a validation.

In some embodiments, any suitable method for validation can be used, including but not limited to K-fold cross-validation, advanced cross-validation, random cross-validation, grouped cross-validation (e.g., K-fold grouped cross-validation), bootstrap bias corrected cross-validation, random search, and/or Bayesian hyperparameter optimization.

In some embodiments, the validation dataset comprises, for each respective validation subject in a plurality of validation subjects, a corresponding plurality of sub-waveforms and an associated label indicating whether or not the subject has a cardiovascular abnormality. In some embodiments, the validation dataset is a hold-out test set obtained from a training dataset.

In some embodiments, the plurality of validation subjects includes at least 100, at least 1000, at least 10,000, or at least 100,000 validation subjects. In some embodiments, the plurality of validation subjects includes no more than 500,000, no more than 100,000, or no more than 10,000 validation subjects. In some embodiments, the plurality of validation subjects includes from 10,000 to 500,000, from 1000 to 10,000, or from 2000 to 20,000 validation subjects. In some embodiments, the plurality of validation subjects falls within another range starting no lower than 100 validation subjects and ending no higher than 500,000 validation subjects.

In some embodiments, the neural network is trained and/or validated using sub-waveforms and/or full-waveform representations of ECGs, using any of the methods disclosed herein, or any substitutions, modifications, additions, deletions, and/or combinations as will be apparent to one skilled in the art.

In some embodiments, the neural network is an ensemble neural network. For instance, in some embodiments, the neural network comprises at least a first trained neural network and a second trained neural network. The first trained neural network is trained, responsive to receiving, as input, sub-waveforms of ECGs for a respective subject, to output a first determination of whether or not the respective subject has a cardiovascular abnormality, and the second trained neural network is trained, responsive to receiving, as input, full-waveforms of ECGs for a respective subject, to output a second determination of whether or not the respective subject has a cardiovascular abnormality. In some such embodiments, the first determination and the second determination are combined to obtain a final determination of whether or not the respective subject has a cardiovascular abnormality.

### Scoring Electrocardiograms and Representations Thereof.

As described above, in some embodiments, the ability to identify clinically relevant features is useful for establishing and administering downstream applications such as treatment strategies to optimize efficacy in improving symptoms and clinical outcomes. Accordingly, in some embodiments, the method 200 further includes scoring an electrocardiogram (e.g., for a respective subject) and/or a representation thereof (e.g., a sub-waveform) based on one or more heartbeat features of the ECG and/or the representation. In some embodiments, the scoring the ECG and/or the representation thereof includes scoring one or more parameters of the neural network to determine an importance of a respective one or more heartbeat features in the ECG and/or the representation.

For instance, referring to Block 232, in some embodiments, the method further comprises using the plurality of parameters to compute a saliency map of at least a subset of the plurality of parameters. In some embodiments, the plurality of parameters is used to compute the saliency map after the neural network is trained to determine a cardiovascular abnormality. Thus, in some such embodiments, the plurality of parameters include a plurality of weights that indicate which features of one or more input ECG representations (e.g., sub-waveform and/or full-waveform) are most significant in making the determination. In other words, a saliency map generally indicates a gradient of the effect of changes in input ECG (e.g., data points corresponding to a respective ECG) on predicted outcome (e.g., determination of cardiovascular abnormality).

In some embodiments, the saliency map is computed for each respective sub-waveform in a set of sub-waveforms. In some embodiments, the saliency map is computed for each respective full-waveform representation in a set of full-waveform representations (e.g., full-length ECGs).

In some embodiments, the saliency map includes the positive and negative values (e.g., positive and negative effects of input on outcome, such as inputs that contribute respectively to a prediction of presence or absence of cardiovascular abnormality). In some such embodiments, the positive and negative portions of the saliency map are converted to absolute values. In some embodiments, the values obtained from the saliency map are normalized (e.g., min-max normalized). In some such embodiments, the normalized values of the saliency map fall within a range of 0 to 1.

Saliency maps are known in the art, as described, for example, in Shrikumar et al., “Learning Important Features Through Propagating Activation Differences,” Proceedings of the 34th International Conference on Machine Learning, Sydney, Australia, PMLR 70, 2017, which is hereby incorporated herein by reference in its entirety.

In some embodiments, the computing the saliency map further includes performing an alignment of a set of input ECG representations (e.g., sub-waveforms and/or full-waveform representation).

In some embodiments, the alignment of a respective set of ECG representations comprises, for each respective ECG representation in the set of ECG representations, identifying a feature location of a corresponding first instance of an ECG heartbeat feature, thus identifying a set of feature locations. The set of ECG representations is aligned along their respective feature locations, thus obtaining a set of aligned ECG representations.

In some embodiments, the alignment further includes, for each respective aligned ECG representation in the set of aligned ECG representations, determining a respective start location (e.g., left edge of the ECG representation) and a respective end location (e.g., right edge of the ECG representation). Each respective aligned ECG representation, other than the aligned ECG representation with the left-most start location, is left-padded at the respective start location, and each respective aligned ECG representation, other than the aligned ECG representation with the right-most end location, is right-padded at the respective end location, thereby obtaining a set of aligned ECG representations having coincident start locations, feature locations, and end locations.

In some embodiments, the respective set of ECG representations is a set of sub-waveforms, and the method comprises aligning the set of sub-waveforms using any of the above methods or a combination thereof. In some embodiments, the respective set of ECG representations is a set of full-waveform representations, and the method comprises aligning the set of full-waveform representations using any of the above methods or a combination thereof.

In some embodiments, the first heartbeat feature is a QRS complex, a P-value, a PR segment, a T-wave, and/or an ST segment. In some embodiments, the feature location is any measurable point on the respective heartbeat feature, including, but not limited to, a start location of the respective heartbeat feature, a midpoint of the respective heartbeat feature, an end location of the respective heartbeat feature, and/or a weighted midpoint of the respective heartbeat feature.

In some embodiments, the left-padding comprises padding the left end of the respective ECG representation with null or zero elements. In some embodiments, the right-padding comprises padding the left end of the respective ECG representation with null or zero elements.

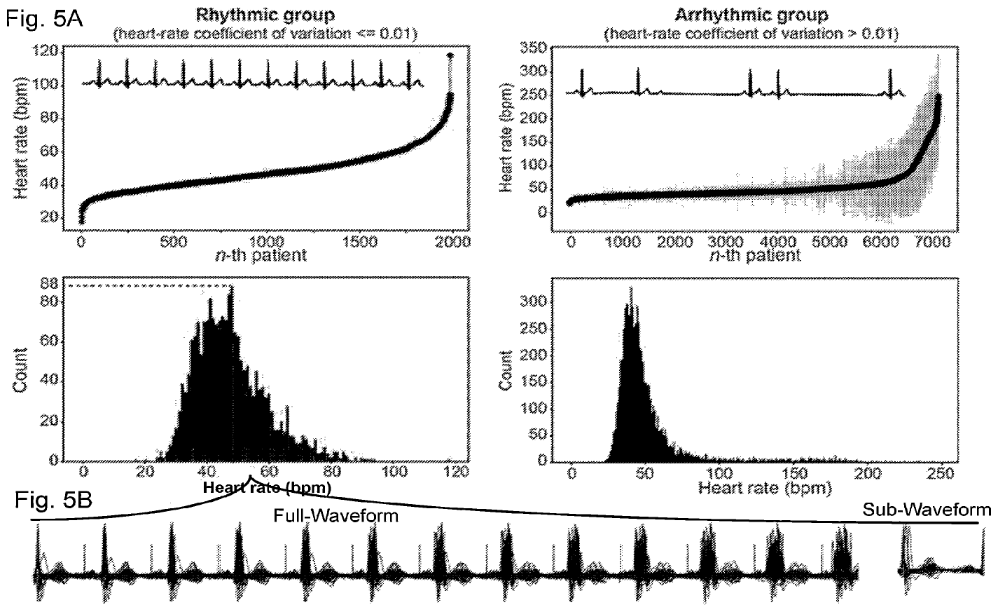

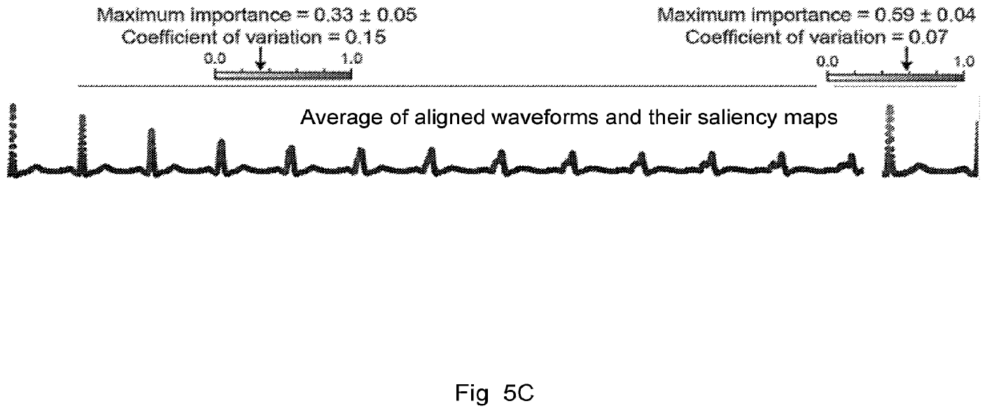

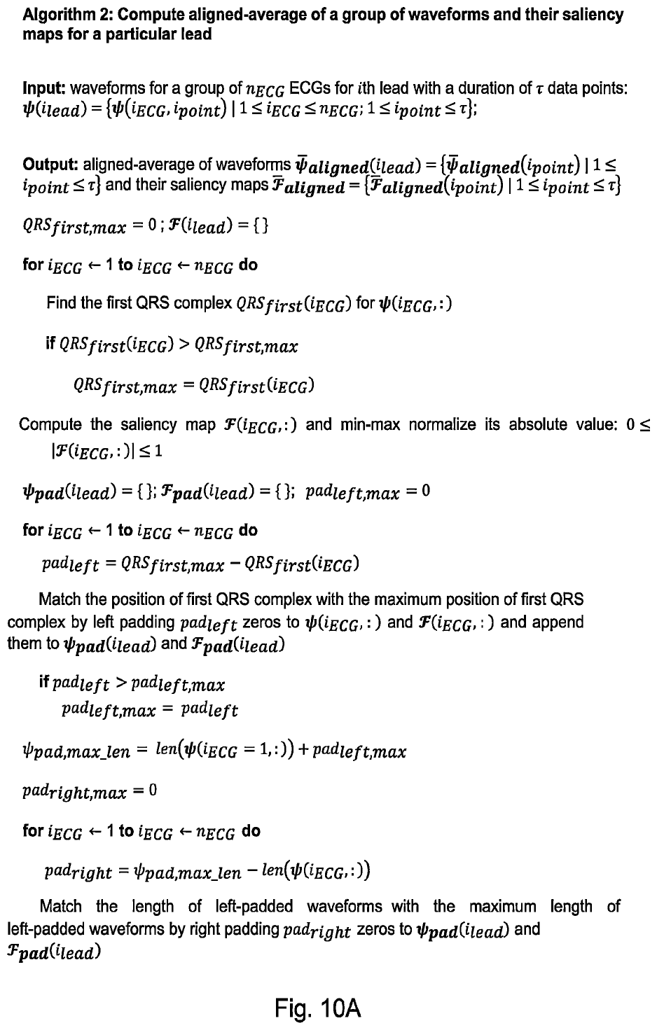

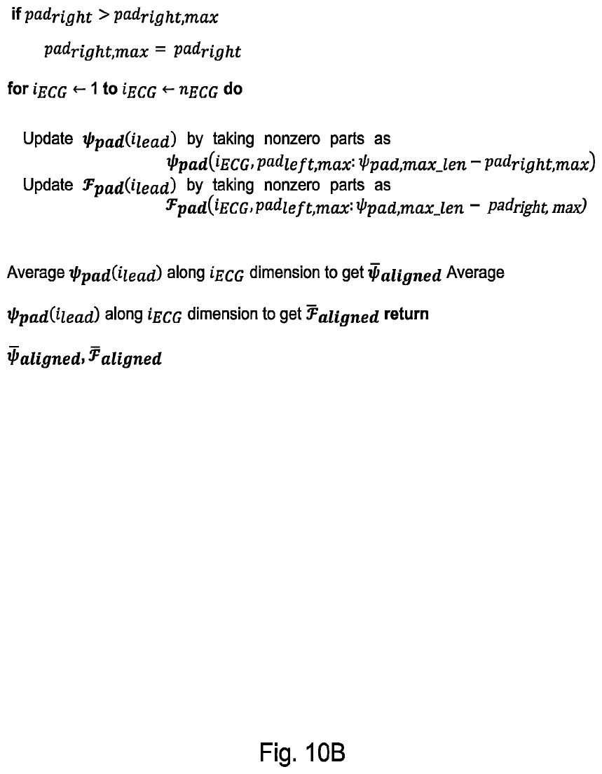

For example, FIGS. 10A-B illustrate algorithmic steps by which ECG representations are aligned. The exemplary algorithm includes, for each respective waveform (e.g., ECG representation) in a plurality of waveforms, identifying the first QRS complex in the respective waveform and computing a respective saliency map for the respective waveform, where the values of the respective saliency map are converted to absolute values and min-max normalized. Waveforms are then matched such that they are aligned along the first QRS complex. Each respective waveform is then left-padded and right-padded such that the start and end points of each respective waveform are coincident with the left-most and right-most waveforms, based on the alignment of the matched first QRS complexes, and such that each waveform in the plurality of waveforms has the same length. ECG representations (e.g., sub-waveform and full-waveform) that are aligned in accordance with some embodiments of the present disclosure are illustrated, for example, in FIG. 5B.

In some embodiments, where a respective saliency map is computed for each respective ECG representation (e.g., sub-waveforms and/or full-waveform representation) in a set of ECG representations, the resulting set of saliency maps for the set of ECG representations is averaged. In some embodiments, where the computing the saliency map further includes performing an alignment of the set of input ECG representations, the set of aligned ECG representations are averaged.

Accordingly, in some embodiments, computing a saliency map comprises generating an aligned average of the set of ECG representations (e.g., sub-waveforms and/or full-waveform representations) and the corresponding set of saliency maps for the set of ECG representations. In some embodiments, as provided in FIG. 10B, the averaging comprises, for each respective ECG representation in the set of ECG representations, removing any portion of the waveform that is aligned with a zero or null element in a portion of any other waveform in the set of aligned ECG representations. Thus, in some embodiments, only the nonzero, overlapping portions of waveforms in the set of aligned ECG representations are used for the averaging.

In some embodiments, a respective one or more saliency maps for a respective one or more ECG representations (e.g., sub-waveforms and/or full-waveform representations) are superimposed onto the waveform(s) of the respective one or more ECGs. In some embodiments, the one or more saliency maps are superimposed on an average and/or alignment of the one or more ECG representations. Accordingly, as illustrated in FIG. 5C, in some embodiments, a respective saliency map is superimposed onto an averaged representation of a corresponding plurality of aligned ECG representations (e.g., sub-waveform and/or full-waveform representations), for at least one lead in the plurality of leads. In some such embodiments, the superimposing of saliency maps onto ECG representations allows for the visualization of the relative importance of heartbeat features (e.g., a QRS complex, a P-value, a PR segment, a T-wave, and/or an ST segment) to clinical outcomes and assessment of ECGs.

In some embodiments, the method comprises using the plurality of parameters to compute a saliency map for a respective one or more ECG representations and removing the positive values from the saliency map, thus generating a negative saliency map (e.g., including only negative effects of input on outcome, such as inputs that contribute respectively to a prediction of absence of cardiovascular abnormality). In some embodiments, the method comprises using the plurality of parameters to compute a saliency map for a respective one or more ECG representations and removing the negative values from the saliency map, thus generating a positive saliency map (e.g., including only positive effects of input on outcome, such as inputs that contribute respectively to a prediction of presence of cardiovascular abnormality). In some such embodiments, the values of the saliency map are converted to absolute values. In some embodiments, the values obtained from the saliency map are normalized (e.g., min-max normalized). In some such embodiments, the normalized values of the saliency map fall within a range of 0 to 1.

In some embodiments, the method further includes using a saliency map for a respective one or more ECG representations to compute an importance score for at least a heartbeat feature in a plurality of heartbeat features in the one or more ECG representations. In some embodiments, each respective importance score shows the contribution of the respective heartbeat feature to the predicted outcome or probability (e.g., the higher the score, the higher the contribution to predicted probability). For instance, development of a scoring system to quantify the importance of heartbeat features has potential use as an informatics tool for clinical decision making.

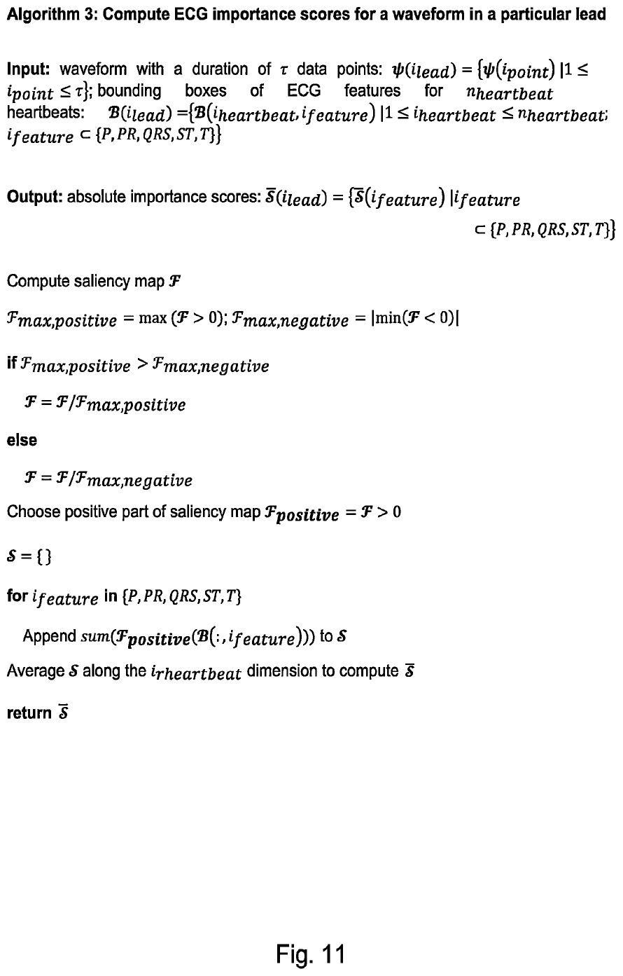

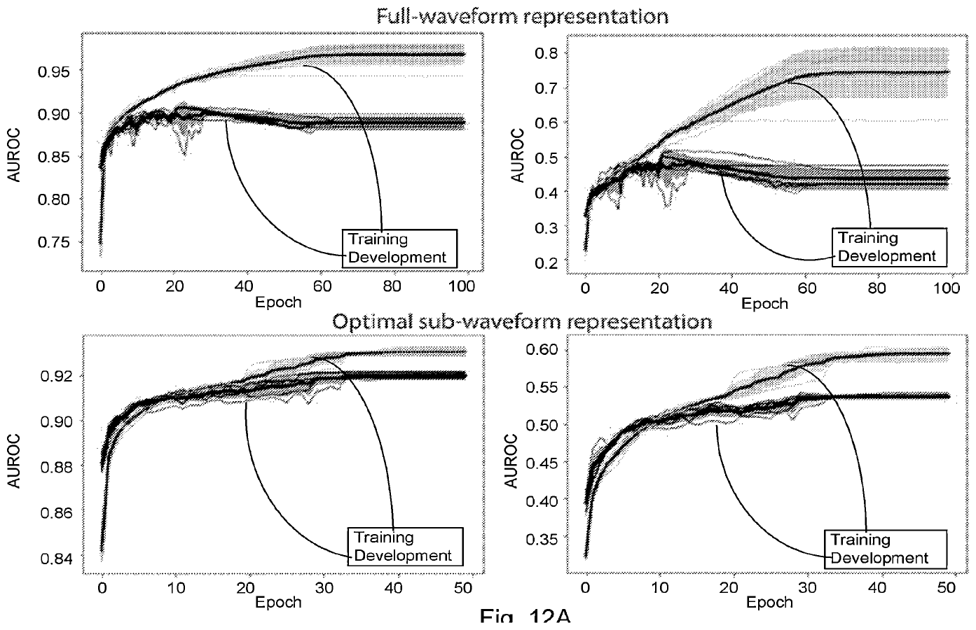

Accordingly, as detailed in the exemplary algorithm in FIG. 11, in some embodiments, the method comprises generating a saliency map (e.g., positive and/or negative) for a respective ECG representation (e.g., waveform). In some such embodiments, values for each instance of each respective heartbeat feature (e.g., selected from QRS complex, P-value, PR segment, T-wave, and ST segment) are obtained from the saliency map and averaged across the plurality of instances of the respective heartbeat feature, thus generating an absolute importance score. For instance, in some embodiments, the absolute importance score is obtained for a full-waveform representation (full) [symbol] _{full} and/or a sub-waveform representation (sub) [symbol] _{sub}. Normalized scores for each representation are calculated as follows:

$$\\ {scores}_{full} = \frac{{full}_{}}{max ( {\sum }_{{i}_{feature}} {full}_{} , {\sum }_{{i}_{feature}} {sub}_{} )} , and$$

$$\\ {scores}_{sub} = \frac{{sub}_{}}{max ( {\sum }_{{i}_{feature}} {full}_{} , {\sum }_{{i}_{feature}} {sub}_{} )} ,$$

where the absolute scores are normalized by the maximum of the summation of [symbol] _{full} over all features and the summation of [symbol] _{sub} over all features. This normalization allows quantitative visualization of the differences between the two representations (where the plurality of sub-waveforms corresponding to a given full-length waveform representation of a given lead are collectively considered a sub-waveform representation for the given full-length waveform).

In some embodiments, as illustrated in FIG. 6C, a respective one or more importance scores for a respective one or more heartbeat features are superimposed onto the waveform(s) of a respective one or more ECG representations (e.g., sub-waveform and/or full-waveform). In some embodiments, the one or more importance scores are superimposed on an average and/or alignment of the one or more ECG representations. In some embodiments, the respective one or more importance scores are superimposed onto an averaged representation of a corresponding plurality of aligned ECG representations (e.g., sub-waveforms and/or full-waveform representation), for at least one lead in the plurality of leads. In some such embodiments, the superimposing of importance scores onto heartbeat features displayed in a visual representation of an ECG allows for the identification of the relative importance of heartbeat features (e.g., a QRS complex, a P-value, a PR segment, a T-wave, and/or an ST segment) to clinical outcomes and assessment of ECGs.

In some embodiments, as described above and as illustrated in FIG. 6C, the visual representation of the ECG further comprises one or more annotations. In some such embodiments, the one or more annotations indicates the location of one or more heartbeat features (e.g., a QRS complex, a P-value, a PR segment, a T-wave, and/or an ST segment). In some embodiments, the one or more annotations indicates a specific cardiovascular abnormality-related feature (e.g., a specific LVD-related feature). In some embodiments, the one or more annotations indicates a non-specific cardiovascular abnormality-related feature (e.g., a non-specific LVD-related feature).

For instance, in some embodiments, the method further comprises using a visual examination of an ECG to determine an importance of a respective heartbeat feature. In some embodiments, the method further comprises using a visual examination of a saliency map to determine an importance of a respective heartbeat feature. In some such embodiments, the visual examination is performed by a cardiology expert. In some embodiments, the method comprises using the visual examination to confirm and/or validate one or more computed importance scores. In some embodiments, the method comprises using one or more computed importance scores to confirm and/or validate a clinical diagnosis (e.g., a specific and/or a non-specific LVD-related feature) obtained from a healthcare provider such as a clinician.

Thus, in some embodiments, the method comprises superimposing one or more of an annotation, a saliency map, and an importance score onto a corresponding one or more ECG representations, where the one or more ECG representations are optionally aligned and/or averaged.

In some embodiments, any suitable method for identifying and/or scoring important features used in neural network learning are contemplated for use in the present disclosure, in addition or as an alternative to saliency maps.

For example, referring to Block 234, in some embodiments, the method further comprises using an occlusion approach to determine a weighting of affect on the indication for each time point in the plurality of time points for at least one lead in the plurality of leads. Thus, in some embodiments, any of the methods disclosed herein for computing and/or averaging feature importance, aligning waveforms, averaging waveforms, and/or superimposing feature importance into waveforms, as disclosed herein with reference to saliency maps and/or importance scores, are further contemplated for use with an occlusion approach, as will be apparent to one skilled in the art. Accordingly, in some embodiments, the method further includes superimposing the weighting of affect on the indication for each time point in the plurality of time points onto an ECG representation for at least one lead in the plurality of leads. In some embodiments, the method further includes superimposing the weighting of affect on the indication for each time point in the plurality of time points onto an averaged representation of the corresponding plurality of temporally aligned sub-waveforms for the at least one lead in the plurality of leads.

Referring to Block 236, in some embodiments, the occlusion approach is forward pass attribution.

Other methods for identifying important features used in neural network learning are possible, as will be apparent to one skilled in the art. For instance, perturbation-based forward propagation approaches make perturbations to individual inputs or neurons and observe the impact on later neurons in the network. Backpropagation-based approaches propagate an importance signal from an output neuron backwards through the layers to the input in one pass. One such backpropagation-based approach is DeepLIFT. Gradients, deconvolutional networks, and guided backpropagation-based approaches, including saliency maps, use the gradient of the output with respect to pixels of an input image to compute an importance map of the image in the context of image classification tasks. Additional suitable methods include, but are not limited to, layerwise relevance propagation and gradient input-based approaches, integrated gradient-based approaches, grad-CAM, and/or guided CAM. See, for example, Shrikumar et al., “Learning Important Features Through Propagating Activation Differences,” Proceedings of the 34th International Conference on Machine Learning, Sydney, Australia, PMLR 70, 2017, which is hereby incorporated herein by reference in its entirety.

### ADDITIONAL EMBODIMENTS

Another aspect of the present disclosure provides a method for determining whether a subject has a cardiovascular abnormality, the method comprising, at a computer system comprising a memory, obtaining an electrocardiogram of the subject, where the electrocardiogram represents a plurality of time intervals summing to a total time interval, where each time interval in the plurality of time intervals has a first duration and the plurality of time intervals comprises at least 400 time intervals. The electrocardiogram comprises, for each respective lead in a plurality of leads, a corresponding plurality of data points, each respective data point in the plurality of data points representing an electronic measurement at the respective lead at a different time interval in the plurality of time intervals.

The method further includes obtaining, for each respective lead in the plurality of leads, a corresponding plurality of sub-waveforms from the electrocardiogram, each respective sub-waveform in the corresponding plurality of sub-waveforms (i) having a common duration that is less than the total time interval, where the common duration represents a fixed multiple of a reference heartbeat duration, and (ii) offset from a beginning of the electrocardiogram by a unique multiple of a sliding parameter. The corresponding plurality of sub-waveforms for each lead in the plurality of leads is inputted into a neural network, thereby obtaining an indication as to whether the subject has the cardiovascular abnormality, where the neural network comprises a plurality of parameters (e.g., 500 or more parameters).

Another aspect of the present disclosure provides a non-transitory computer readable storage medium, where the non-transitory computer readable storage medium stores instructions, which when executed by a computer system, cause the computer system to perform a method for determining whether a subject has a cardiovascular abnormality. The method comprises obtaining an electrocardiogram of the subject, where the electrocardiogram represents a plurality of time intervals summing to a total time interval, where each time interval in the plurality of time intervals has a first duration and the plurality of time intervals comprises at least 400 time intervals. The electrocardiogram comprises, for each respective lead in a plurality of leads, a corresponding plurality of data points, each respective data point in the plurality of data points representing an electronic measurement at the respective lead at a different time interval in the plurality of time intervals.

The method further includes obtaining, for each respective lead in the plurality of leads, a corresponding plurality of sub-waveforms from the electrocardiogram, each respective sub-waveform in the corresponding plurality of sub-waveforms (i) having a common duration that is less than the total time interval, where the common duration represents a fixed multiple of a reference heartbeat duration, and (ii) offset from a beginning of the electrocardiogram by a unique multiple of a sliding parameter. The corresponding plurality of sub-waveforms for each lead in the plurality of leads is inputted into a neural network, thus obtaining an indication as to whether the subject has the cardiovascular abnormality, where the neural network comprises a plurality of parameters (e.g., 500 or more parameters).

Yet another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors.

Still another aspect of the present disclosure provides a non-transitory computer readable storage medium storing one or more programs configured for execution by a computer, the one or more programs comprising instructions for carrying out any of the methods disclosed herein.

### EXAMPLES

### Example 1 Performance of Sub-Waveform for LVD Prediction

In an example, the performance of sub-waveforms compared to full-waveform representations of ECGs was systematically investigated, in accordance with some embodiments of the present disclosure. The following example describes the improvements gained in deep learning models using the presently disclosed sub-waveforms, provides an interpretation framework for quantifying the importance scores of ECG features, investigates the differences between the interpretation of the two representations, and illustrates the impact of sub-waveforms on disparities in different subgroups.

### Results.

Predictive performance for identification of left ventricular dysfunction (LVD). As a baseline, the left ventricular ejection fraction (LVEF) was predicted for 9,244 patients in a hold-out test set using a full-waveform representation with a 1D CNN with residual blocks, in accordance with some embodiments of the present disclosure (see, for example, Methods: Deep Learning Setup and Evaluation, below). The performance metrics for the full-waveform representation are shown in FIG. 4 and highlighted by the dashed horizontal lines in each of the panels. Full-waveform predictions are close to the results in [25] despite using different datasets and model architectures. For sub-waveforms, a set of systematic experiments were performed to understand the performance of sub-waveforms compared to the full-waveform performance.

FIGS. 4A and 4B illustrate two sets of experiments that were controlled by varying the sliding parameter (e.g., a multiple of reference heartbeat). The sliding parameter was set to a minimum value equal to one reference heartbeat (FIG. 4A) and a maximum value equal to the length of a single sub-waveform. This maximum value was selected because a sliding parameter greater than sub-waveform length results in loss of information from the original full-waveform. For each of the minimum and maximum sliding parameters assessed, the sub-waveform duration was varied from 0.74 to 5.92 seconds (x-axis, bottom). For each sliding parameter (minimum and maximum) and each duration, the prediction performance was assessed for one randomly selected sub-waveform out of the total number of generated sub-waveforms (data points represented as circles) and for all of the sub-waveforms in the maximum generated number of sub-waveforms, where the maximum generated number of sub-waveforms was based on the selected sliding parameter and the selected sub-waveform duration (x-axis, top).

As illustrated in both FIGS. 4A and 4B, predictions obtained using the maximum number of sub-waveforms consistently outperformed those obtained using only one sub-waveform for both the minimum and maximum sliding parameter experimental setups, which was determined to be due, at least in part, to an alignment effect (see, e.g., “Explanation for underlying mechanism of DL improvements,” below). In addition, maximum sliding (FIG. 4B) resulted in lower performance than minimum sliding (FIG. 4A) because the smaller number of generated sub-waveforms weakens the alignment. Optimal performance was observed when the duration of sub-waveforms was set at 1.48 seconds (e.g., each sub-waveform comprises 2 reference heartbeats with a total of 10 generated sub-waveforms) as highlighted by the dashed circles in FIG. 4A. In addition to changes in performance, changes in uncertainties were also observed. In particular, sub-waveform predictions exhibited smaller uncertainties compared to full-waveform predictions. To better understand the variabilities in each respective prediction, the coefficient of variation (CV) was used, where the coefficient of variation represents the ratio of a standard deviation (uncertainty) to a mean. For the full-waveform, the CV was 0.010 for AUROC and 0.094 for AUPRC, whereas these values for the optimal sub-waveform were reduced to 0.001 and 0.017. Controlling and minimizing these uncertainties can have important implications for realizing precision medicine with novel AI technologies.

Explanation for underlying mechanism of DL improvements. To explain the above deep learning results, an explanation framework was developed to directly show how generating sub-waveforms for use as inputs to a neural network (e.g., “heartbeat alignment”) improves performance over using full-waveform representations. In ECG data, heart rate is predicted by measuring the time between heartbeats. Since each ECG captures several heartbeats (for instance, 13 heartbeats in the reference full-waveform representation), the predicted heart rate for each ECG was averaged across all the heartbeats in the respective ECG. Using heart rate variability (quantified by CV for each predicted heart rate), the ECG data was thus divided into two groups: rhythmic (CV ≤0.01) and arrhythmic (CV >0.01). The statistical plots (heart rate for each patient and count of ECGs per heart rate) for these two groups are shown in FIG. 5A. Notably, the reference heart rate of 78 bpm falls within the 95th percentile of rhythmic and arrhythmic ECGs.

The rhythmic group was selected in order to better understand the impact of heartbeat alignment on prediction performance. Although not limited to any one theory of operation, this selection arose in part from potential difficulties in aligning arrhythmic ECGs, due to the random distribution of heartbeats across the full-waveform. Accordingly, the subset of rhythmic ECGs corresponding to the most prevalent heart rate (e.g., the heart rate with the highest count) was selected for further statistical investigation, the subset comprising 88 ECGs with a heart rate of 48 bpm. For this example, statistical analysis was performed using lead I ECGs, as lead I is commonly used in wearable devices [27]. However, in some embodiments, similar analysis can be performed using ECGs obtained from any other lead as disclosed herein. Lead-I waveforms were left-aligned using signal processing techniques (see, for example, Methods: Explanation Framework, below). The aligned waveforms are shown in FIG. 5B for both the full-waveform representations and sub-waveforms. For each of the 88 waveforms, a saliency map that shows the gradient of predicted outcome with respect to the changes in input ECG was calculated [28]. Generally, for a binary outcome, the positive and negative values of a saliency map show the contribution of features to positive and negative outcomes, respectively. In order to explore the impact of heartbeat alignment on all features, both positive and negative portions of the saliency map were retained and the min-max normalization of the absolute values of the saliency map were used. Aligned waveforms and their saliency maps were then averaged, for both full-length representations and sub-waveforms, as illustrated in FIG. 5C.

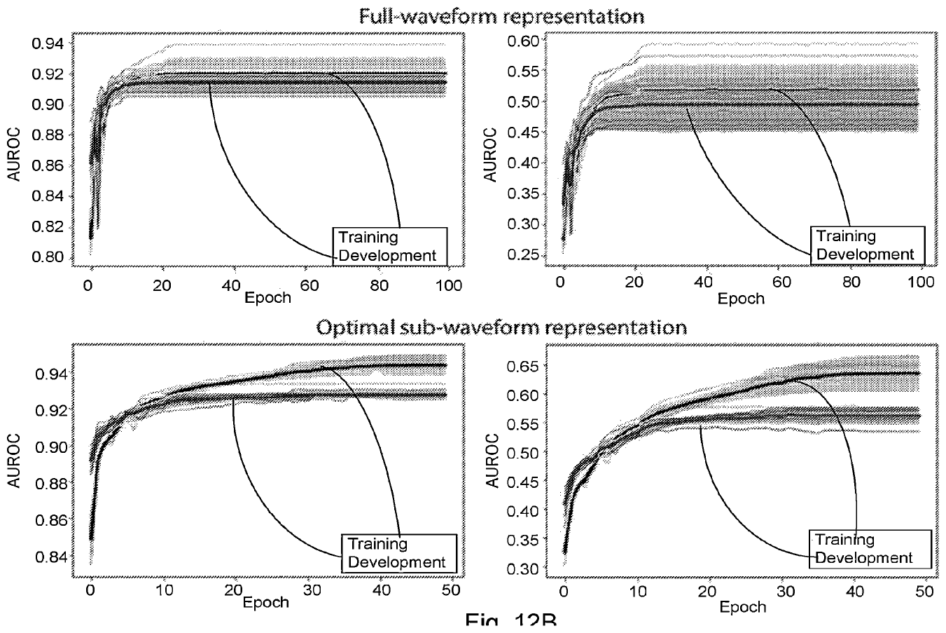

FIG. 5C illustrates that by changing the full-waveform representation to the optimal sub-waveform, the averaged maximum importance increased from 0.33 to 0.59 and the averaged CV decreased from 0.15 to 0.07. This observation is remarkable as it directly shows the enhancement of performance due to the generation of rhythmic sub-waveforms for input (e.g., heartbeat alignment). For example, in the full-waveform representation of rhythmic ECGs, the repeated heartbeats that are similar in shape introduce redundancies in the learned weights; the sub-waveform improves this deterioration in learning by aligning heartbeats (e.g., collapsing repeated heartbeats from a single ECG into a plurality of individual training examples). In particular, as shown in FIGS. 12A and 12B, full-waveform inputs caused significant overfitting in shallower neural networks (FIG. 12A: shallower neural network, top panels) compared to both sub-waveform inputs (FIG. 12A: shallower neural network, bottom panels) and deeper neural networks (FIG. 12B: deeper neural network, top panels). Conversely, sub-waveform inputs were relatively stable in terms of overfitting with respect to changes in neural network depth (FIG. 12A: shallower neural network, bottom panels; and FIG. 12B: deeper neural network, bottom panels). Notably, the performance results obtained using sub-waveforms further capture the features of arrhythmic ECGs, in addition to rhythmic ECGs, because the duration of the optimal sub-waveform corresponds to the maximum observed heart rate of 156 bpm, which falls within the 99th percentile of arrhythmic ECGs.

Interpretation of ECG features. In some embodiments, the black-box nature of DL precludes understanding what features in ECG waveforms are meaningful and important for a particular task [29-31]. Despite the high performance that DL models for ECGs can achieve in a variety of tasks, interpreting the most relevant features may still present a challenge, thus limiting their use in clinical workflows [3, 4, 16]. Therefore, identifying clinically relevant features provides opportunities for tailoring further workup and treatment strategies to optimize efficacy in improving symptoms and clinical outcomes [32]. As shown in FIG. 6A, in some embodiments, a respective waveform is characterized by 5 ECG features, including P-wave, PR segment, QRS complex, ST segment, and T-wave [1]. These features provide significant clinical information for assessing ECGs. Advantageously, the present disclosure provides a scoring system that allows a user to quantify the importance of ECG features for downstream use as an informatics tool for clinical decision making.

A full-waveform model with AUROC=0.918 and AUPRC=0.473 and an optimal sub-waveform model with AUROC=0.926 and AUPRC=0.578 were selected for ECG feature interpretation analysis. For both full-length representations and sub-waveforms, the predicted probabilities were extracted, and the optimal decision threshold was determined (dashed line; threshold=0.08). The prediction probabilities for each patient and the corresponding confusion matrices are shown in FIG. 6B (left panels: full-waveform; right panels: sub-waveform). Due to the higher predictive performance of sub-waveforms, patients that were classified as false negative (FN) using full-waveform inputs and true positive (TP) using sub-waveform inputs were selected for further ECG feature analysis. This selection was made in order to highlight potential insights into the clinical relevance of model predictions and how the features of sub-waveforms drive accuracy in predictions (e.g., FN versus TP).

A total of 35 patients were identified as having full-waveform FN predictions and sub-waveform TP predictions, whereas 22 patients were identified as having full-waveform TP predictions and sub-waveform FN predictions. The ECGs were sorted and ranked based on the difference in the predicted probabilities of sub-waveform TP and full-waveform FN. The top five ranked patients (e.g., having the greatest differences in sub-waveform TP and full-waveform FN probabilities) comprised differences in probabilities ranging from 0.55 to 0.20. As illustrated in FIG. 6B, false positive predictions increased by 1.6% while false negative predictions decreased by 11.7% when the sub-waveforms were used, compared to the full-waveform representation. The decrease in FN is notable, as the class imbalance for full-length versus sub-waveform was consistent and the percent positive was 0.076. For instance, in a hold-out test set, there were only 703 positive patients and 8,543 negative patients. These results are promising, especially for emerging AI technologies in healthcare that strive to (i) increase sensitivity by accurately identifying all positive patients (i.e., low FN) and (ii) increase specificity by reducing the number of false positives that can impose additional costs and alarm fatigue (i.e., low FP) [33].

As illustrated in FIGS. 6C and 8A-E, out of the top five retrieved patients, the ECGs for patients I, III-V are clearly rhythmic. But the rhythmic pattern for patient II is less clear. To better understand the characteristics of these ECGs, assessments performed by cardiac electrophysiology experts confirmed that patients I, III-V have overall sinus rhythms [34], while patient II has a vector of pacing that is consistent with cardiac resynchronization therapy (CRT), suggesting that the patient likely has underlying LVD as CRT is normally utilized in patients with heart failure and left bundle branch block to restore left and right ventricular synchrony or in patients with LVD who have a high burden of pacing [35]. This clinical validation reconfirms the substantial impact of sub-waveforms on neural network learning for rhythmic ECGs, as described above.

For each patient, positive saliency maps for both full-length and sub-waveforms were calculated and are presented in FIG. 6C (see, e.g., Methods: Interpretation Framework). Unlike the positive and negative saliency maps described above for calculating maximum importance of representations to predictions, the positive saliency maps incorporate only the positive portion of the saliency map (e.g., the portion that contributes to the positive outcome), in order to highlight patients with positive outcomes. The ECGs of patients I-V were annotated with the five ECG features (e.g., P-wave, PR segment, QRS complex, ST segment, and T-wave) by cardiac electrophysiology experts using the full-waveform representation across all heartbeats and leads. Annotated ECGs for patients I-V are illustrated in FIGS. 8A-E, respectively. Additionally, sub-waveforms for each of patients I-V were annotated using the same annotations as applied to the full-waveform representations, for consistency and to provide fair comparisons. For each ECG feature, a normalized score was calculated using the saliency map and annotations (see, e.g., Methods: Interpretation Framework). Each score shows the contribution of the ECG feature to the predicted probability, where higher scores indicate a higher contribution to predicted probability. FIG. 6D illustrates that sub-waveforms transform and highlight the important ECG features in each of the patients.

To better understand the meaning of the scores and connect deep learning predictions of important features with cardiologists' predictions, specific and nonspecific LVD-related features in the ECGs were further assessed by cardiac electrophysiology experts. For instance, whereas expert assessment identified a widened and notched QRS complex in patient I, in agreement with the calculated importance scores illustrated in FIG. 6D, this feature was determined to be abnormal depolarization and nonspecific for LVD. Additionally, ST depression in patient III was determined by expert assessment to be a nonspecific feature for LVD in association with myocardial ischemia, confirming the importance of the ST segment (importance score of 0.50) as calculated by deep learning using sub-waveforms. In contrast, deep learning models trained on full-waveform representations predicted a greater importance for the P-wave, with a score of 0.32.

Only patient II was identified by expert assessment as having specific features for LVD, mainly due to the paced vector of QRS complexes being consistent with CRT pacing (cardiac resynchronization therapy). A deeper investigation of the corresponding saliency maps for patient II (either for full-waveform or sub-waveform) was performed to search for this feature. The saliency maps visually highlighted a clinically irrelevant stimulation artifact as the most important feature and revealed that the ECG of patient II lacks a P-wave and PR segment. Determining the extents of QRS complex, ST segment, and T-wave is nontrivial for a nonexpert. However, the results illustrated in FIG. 6D show that the importance scores accurately predict the paced QRS complex as the most important feature. The QRS complex has a score of 0.6 in the full-waveform representation, which changes to 0.8 in the sub-waveform case by suppressing the ST-segment score of 0.2. This clinically meaningful enhancement is the leading cause for pushing the predicted probability of LVD for patient II from 0.07 to 0.47 (FIG. 6C).

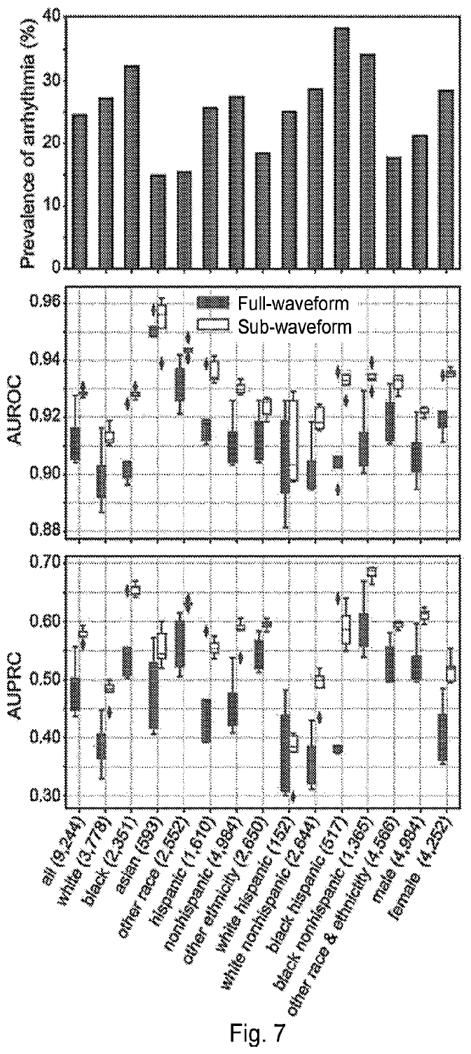

Impact of ECG waveform representation on subgroups. Another concern for DL in healthcare is the potential disparity that predictive capabilities of these models are not fair to all subpopulations that negatively impact certain subgroups in society [36]. To mitigate these biases, fairness researchers have proposed algorithmic techniques to minimize disparities in predictions (i) across subgroups (called group fairness) and (ii) within a subgroup (called individual fairness) [37]. In the present study, a potential source of bias for predictive performance is the possibility of a higher number of rhythmic to arrhythmic ECGs in each subgroup [38]. This bias can be mitigated by optimizing the waveform representation. In FIG. 7, the prevalence of arrhythmia for 14 racial, ethnical, and gender subgroups in a hold-out test set is shown. The AUROC and AUPRC for these subgroups are shown. For both representations, disparities across subgroups were observed, which could be mitigated using group fairness techniques. Regarding individual fairness, it was observed that for each subgroup, DL uncertainty using the sub-waveforms were much smaller than when using the full-waveform representation. These results illustrate that sub-waveform representation can help to mitigate the disparities in DL predictions within a subgroup with a lower prevalence of arrhythmia (e.g., higher number of rhythmic ECGs).

### DISCUSSION

The foregoing results illustrate that sub-waveforms of ECGs enhance predictions obtained from deep learning methodologies. Compared to traditional full-waveforms, rearranging ECG waveforms into sub-waveforms confer an added control that provides many opportunities for enhancing DL modeling capabilities. In the above example, to investigate the impact of the sub-waveforms as a form of input data transformation, all parameters of the neural network in which it was used were kept fixed [39]. Thus, the observed gains in performance compared to the traditional full-waveform representation is indicative of the transformation of the input data to the sub-waveforms.

Investigation of the performance of sub-waveforms for identifying LVD revealed improvements in performance metrics (2% and 20% increase for AUROC and AUPRC) and, importantly, reductions in uncertainties (90% and 80% decrease for AUROC and AUPRC). As uncertainty is a source of bias [40] and due to observed variations in prevalence of arrhythmia across subgroups, the impact of data representation on subgroups was further examined. For instance, because predictions for a subgroup with low prevalence of arrhythmia (e.g., a higher number of rhythmic ECGs) can be biased due to full-waveform redundancies, in some embodiments, they can benefit from the use of sub-waveform due to significantly reduced uncertainties for rhythmic ECGs.

In some embodiments, the observed change in uncertainty is a direct consequence of ECG waveform representations and their impact on DL optimization stability. As described above, subgroup analysis illustrated the importance of the use of sub-waveforms for individual fairness, which primarily aims at providing homogenous predictions for individuals within the same subgroup [41]. Indeed, the foregoing results revealed that, in some implementations, waveform representation controls the fluctuations in DL predictions and thus can be used as a bias mitigation tool. Investigations on individual fairness in this context can help to achieve the goal of operationalizing fairness for new ECG-AI systems that are needed in the art [42].

The foregoing results further illustrate that, in some embodiments, assigning different weights to similarly shaped heartbeats in the full-waveform representation of each lead impedes the neural network from finding optimal features across similar ECG examples. For example, inputs comprising full-waveform representations create redundancies that can cause overfitting and deteriorate learning. However, by using sub-waveforms, assigning different weights to similar heartbeats is less likely because of the reduced number of heartbeats and increased number of aligned sub-waveforms that can also be treated as new training examples per patient (e.g., subject). The explanation analysis (see, e.g., the above section entitled, “Explanation for underlying mechanism of DL improvements”) provided evidence for this improved learning by the process of aligning heartbeats in the sub-waveform, thereby providing better localization of important features with reduced uncertainty.

To provide clinical interpretation of these predictions, a novel scoring system for quantifying the DL importance of ECG features was developed. Although visualizing saliency maps that highlight only important ECG data points are known in the art, the above example illustrates the development of a scoring system for ECG-DL modeling. This technique leverages the saliency map to illustrate the importance of a collection of data points that represent one or more ECG features, rather than only the importance of individual data points. Accordingly, the interpretation framework (see, e.g., the above section entitled, “Interpretation of ECG features”) showed that sub-waveforms generally perform better at localizing and highlighting important features that result in a higher prediction probability.

To verify the predicted scores, the framework was applied to a paced ECG with specific features of LVD for which determining the importance of ECG features by visual examination of saliency maps was difficult. The predicted importance scores were qualitatively in high agreement with the predictions of a cardiac electrophysiology team, such that the predicted importance scores could be used to quantify, confirm, and connect clinical predictions that would have been otherwise difficult to interpret. In addition to clinical validation, the interpretation framework also assists clinicians in cases for which ECG features are nonspecific. For example, the scoring system was used to assist clinicians in determining the relative importance of otherwise nonspecific findings such as widened QRS complex or ST segment depression.

In summary, the foregoing results illustrate that sub-waveforms enhance the DL modeling of ECG waveforms. Sub-waveforms can perform better than traditional full-waveform representations for the identification of LVD. An example explanation was provided for the underlying mechanism for DL improvements gained by aligning sub-waveforms. Moreover, an example interpretation framework was provided and validated against cardiologists' predictions. Finally, sub-waveforms provide advantages in mitigating uncertainties within subgroups.

### EXAMPLE METHODS

Sub-waveform Algorithmic Development. By taking advantage of rhythmic pattern of ECG waveforms, full-waveforms were discretized using rhythmic points into heartbeats. For instance, in some embodiments, each heartbeat is the smallest unit of a waveform. FIG. 3B visualizes this discretization for lead I. The rhythmic discretization allows extraction of sub-waveforms at rhythmic points with a duration that is a multiple of the heartbeat. To diversify the sub-waveforms, a “sliding” parameter is introduced, where the sliding parameter controls where the sub-waveform should originate and how many sub-waveforms are created depending on the duration of sub-waveforms. For example, FIG. 3C illustrates how a sub-waveform with 3-heartbeat duration and 2-heartbeat sliding results in 5 sub-waveforms that are generated from the original full-waveform. Once the sub-waveforms are created, the new representation for lead I is formed as shown in FIG. 3D. By applying the same procedure used for lead I for all leads, the final sub-waveform for 8 leads is derived and shown in FIG. 3E. For numerical implementation of the sub-waveform, consider a reference ECG that has 13 heartbeats and has a heart rate of 78 bpm, which is around the average heart rate of adults [49]. A reference heartbeat of 0.74 seconds is used for creating sub-waveforms of all ECGs (e.g., all ECGs in a plurality of training ECGs and all ECGs used as input to a trained neural network) to ensure the same input length for all ECGs inputted to the DL model. Pseudocode for an algorithm for generating a plurality of sub-waveforms is provided in FIG. 9.

Data Pipeline. A training dataset of 92,446 unique patients was obtained. Characteristics of this training dataset are shown in Table 1. Raw ECGs were stored in Extensive Markup Language (XML) files that included patient and test demographics, diagnostic information, and waveform details. Each waveform was recorded at 500 Hz for a duration of 10 seconds (total of 5000 data points) for 12 leads. The 12 leads were reduced to 8 leads because of linear dependencies of 4 leads on other 8 leads. To build a data pipeline, the waveforms for the cohort of patients were retrieved and stored in Hierarchical Data Format (HDF5) suitable for time series data. Before feeding the waveforms into the neural network, several preprocessing steps were performed. To remove baseline drift that may stem from baseline respiration or lead migration, a median filter (width of 1 second or 500 Hz) was used and ECGs flagged with a “Poor Diagnostic Code” from the confirmed reading were removed. Waveforms were further standardized to have zero-mean and unit-variance.

TABLE 1
Dataset for identifying left ventricular dysfunction (LVD)
by predicting left ventricular ejection fraction (LVEF) from
ECG waveforms of 92,446 unique patients. For each patient,
the respective ECG was linked to the nearest-date echo report,
from which the measured LVEF values were taken as ground truth.
LVEF ≤ 35% represented the positive outcome (low LVEF)
and EF > 35% represented the negative outcome (high LVEF).

| | | |
|---|---|---|
| Training | Development | Test |

Dataset characteristics

| | | | |
|---|---|---|---|
| No. patients | 73,956 | 9,246 | 9,244 |
| Percent positive | 7.6 | 7.6 | 7.6 |

Race (%)

| | | | |
|---|---|---|---|
| White | 41.2 | 41.6 | 40.9 |
| Black | 25.1 | 25.0 | 25.4 |
| Asian | 6.6 | 6.6 | 6.4 |
| Other | 27.1 | 26.8 | 27.3 |

Ethnicity (%)

| | | | |
|---|---|---|---|
| Hispanic | 18.4 | 17.9 | 17.4 |
| Non-Hispanic | 53.1 | 53.4 | 53.9 |
| Other | 28.5 | 28.6 | 28.7 |

Gender (%)

| | | | |
|---|---|---|---|
| Male | 54.9 | 54.7 | 53.9 |
| Female | 45.1 | 45.2 | 46.0 |

Age (mean ± standard deviation)

| | | | |
|---|---|---|---|
| — | 63.4 ± 15.1 | 63.2 ± 14.8 | 63.5 ± 15.1 |

In the data pipeline, the ground truth labels were set to LVEF values extracted from echo reports [20, 21] and predicted using the formula: LVEF (%)=(EDV−ESV)/EDV, where EDV and ESV show end-diastolic volume and end-systolic volume. Thus, LVEF values have a continuous range that is suitable for a regression task but that, in some embodiments, can be converted to a binary classification task that is also useful for clinical decision making. As suggested by [25], LVEF ≤35% was selected as the positive outcome (low LVEF) and LVEF >35% (high LVEF) was selected as the negative outcome. Each ECG was linked to the nearest-date echo report. Because the dataset included one ECG per patient, all ECG-echo pairs were unique.

Deep Learning Setup and Evaluation. A deep neural network (DNN) architecture was used for obtaining predictions. See, e.g., reference [9]. The DNN takes preprocessed waveforms as inputs and outputs a binary prediction. Each ECG was represented as a 1D waveform with 8 leads as channels. The neural network included 26 layers and utilized residual connections to make the optimization more effective by avoiding exploding or vanishing gradients [50]. For instance, the neural network included 3 convolutional layers followed by 11 residual blocks (2 convolutional layers per block) and 1 dense layer. After each convolutional layer, a batch normalization was applied to improve learning and a Rectified Linear Unit (ReLU) was implemented to activate the nonlinearities in the network [51]. A dropout regularizer was also used to minimize overfitting in training [52]. The final layer included a fully connected layer followed by a sigmoid function. For training, weights were randomly initialized as described in [53]. The Adam optimizer was used for backpropagation using an initial learning rate of 0.005 and default parameters β!=0.9 and β″=0.999 [54]. A scheduler was used to decay the learning rate by a factor of 0.1 if a metric was not improved for a few epochs.

A development set was used for evaluating the model during training and a hold-out test set was used for reporting the final results. DL models were trained, in accordance with some embodiments of the present disclosure, to classify LVEF severity using ECGs of 73,956 patients for training and ECGs of 9,244 patients for development (e.g., validation). Performance was evaluated using two metrics: area under receiving operating characteristic (AUROC) and area under precision-recall curve (AUPRC). Specifically, performance for 9,244 patients in a hold-out test set was reported. For quantitative evaluation of the models, two curves were calculated: Receiver Operating Characteristic (ROC) and Precision Recall Curve (PRC). Two metrics calculated from these two curves were then reported: Area Under ROC (AUROC) and Area Under PRC (AUROC) as shown in FIGS. 4A-B. For each final prediction, the predictions of 5 independent DL runs with randomly initiated weights were averaged. For bootstrapping, one-randomly selected sub-waveform for each run was extracted. Reported uncertainty for each prediction was calculated as the standard deviation of the 5 bootstrapped predictions.

Explanation Framework. FIG. 9 illustrates the algorithmic steps for the explanation framework. To align waveforms (e.g., full-waveform representations and/or sub-waveforms), the first QRS complexes for each waveform in the plurality of waveforms are matched by left padding and the waveforms are then right-padded to match the length of the left-padded waveforms. In some embodiments, only the nonzero overlapping portions of all the waveforms were used for averaging.

Interpretation Framework. FIG. 11 illustrates the algorithm for calculating importance scores for of ECG features. In the algorithm, bounding boxes are determined a priori and confirmed by clinicians. For example, FIGS. 8A-E illustrate example ECGs annotated with bounding boxes for five top-ranked patients. Bounding boxes were applied consistently for both full-waveform representations and sub-waveforms to make the comparisons reasonable.

Using the interpretation algorithm, the absolute importance scores [symbol] _{full} and [symbol] _{sub} were calculated for full-waveform representations and sub-waveforms. Normalized scores for each representation were calculated as follows:

$$\\ {scores}_{full} = \frac{{full}_{}}{max ( {\sum }_{{i}_{feature}} {full}_{} , {\sum }_{{i}_{feature}} {sub}_{} )}$$

$$\\ {scores}_{sub} = \frac{{sub}_{}}{max ( {\sum }_{{i}_{feature}} {full}_{} , {\sum }_{{i}_{feature}} {sub}_{} )}$$

where the absolute scores were normalized by the maximum of the summation of [symbol] _{full} over all features and the summation of [symbol] _{sub} over all features. This normalization allows quantitative visualization of the differences between the full-length and sub-waveform.

Clinical Interpretation of ECGs. For evaluating the clinical relevance of the interpretation results, cardiac electrophysiology experts were consulted. They annotated the extracted ECGs for the 5 top-ranked patients (FIGS. 8A-E). They confirmed that all 5 patients had a regular rhythm. Specifically, all the patients had sinus rhythms except patient II, whose ECG indicated use of a pacemaker for cardiac resynchronization therapy (CRT). The cardiac electrophysiology team identified the specific and nonspecific LVD-related ECG features. Out of the top 5 patients shown in FIG. 6C, only patient II was determined to have specific features of LVD, which were paced QRS complexes with a paced QRS vector consistent with CRT. In terms of nonspecific features, patient I was identified as having a wider, notched QRS complex in lead I, indicating abnormal conduction and possible association with LVD. Additionally, patient I exhibited T-wave flattening associated with LVD. Furthermore, examination of the lead II ECG of patient III revealed P-wave axis/morphology flips on the 6th, 8th, and 9th annotated heartbeats, suggesting alternative P-wave origins (e.g., palpitations). These observations support an interpretation that patient III may have atrial tachycardia or other atrial arrhythmias. For instance, relatively large, peaked P-waves can be seen in pulmonary hypertension (p pulmonale). A high burden of tachyarrhythmias can also result in LVD (and right ventricular dysfunction) that improves with treatment of the arrhythmia. However, these characteristics alone do not necessarily predict LVD.

### REFERENCES

1. Goldberger, A. L., Z. D. Goldberger, and A. Shvilkin, Clinical electrocardiography: a simplified approach e-book. 2017: Elsevier Health Sciences.
2. Wagner, P., et al., PTB-XL, a large publicly available electrocardiography dataset. Scientific data, 2020. 7(1): p. 1-15.
3. Somani, S., et al., Deep learning and the electrocardiogram: review of the current state-of-the-art. EP Europace, 2021.
4. Siontis, K. C., et al., Artificial intelligence-enhanced electrocardiography in cardiovascular disease management. Nature Reviews Cardiology, 2021: p. 1-14.
5. LeCun, Y., Y. Bengio, and G. Hinton, Deep learning. nature, 2015. 521(7553): p. 436-444.
6. Yu, K.-H., A. L. Beam, and I S. Kohane, Artificial intelligence in healthcare. Nature biomedical engineering, 2018. 2(10): p. 719-731.
7. Minchole, A., et al., Machine learning in the electrocardiogram. Journal of electrocardiology, 2019. 57: p. S61-S64.
8. Miotto, R., et al., Deep learning for healthcare: review, opportunities and challenges. Briefings in bioinformatics, 2018. 19(6): p. 1236-1246.
9. Hannun, A. Y., et al., Cardiologist-level arrhythmia detection and classification in ambulatory electrocardiograms using a deep neural network. Nature medicine, 2019. 25(1): p. 65-69.
10. Wolk, K. and A. Wolk, Early and remote detection of possible heartbeat problems with convolutional neural networks and multipart interactive training. IEEE Access, 2019. 7: p. 145921-145927.
11. Chauhan, S., L. Vig, and S. Ahmad, ECG anomaly class identification using LSTM and error profile modeling. Computers in biology and medicine, 2019. 109: p. 14-21.
12. He, R., et al., Automatic cardiac arrhythmia classification using combination of deep residual network and bidirectional LSTM. IEEE Access, 2019. 7: p. 102119-102135.
13. Le Guennec, A., S. Malinowski, and R. Tavenard. Data augmentation for time series classification using convolutional neural networks. in ECML/PKDD workshop on advanced analytics and learning on temporal data. 2016.
14. Cui, Z., W. Chen, and Y. Chen, Multi-scale convolutional neural networks for time series classification. arXiv preprint arXiv:1603.06995, 2016.
15. Cao, P., et al., A novel data augmentation method to enhance deep neural networks for detection of atrial fibrillation. Biomedical Signal Processing and Control, 2020. 56: p. 101675.
16. Hong, S., et al., Opportunities and challenges of deep learning methods for electrocardiogram data: A systematic review. Computers in Biology and Medicine, 2020: p. 103801.
17. Huang, J., et al., ECG arrhythmia classification using STFT-based spectrogram and convolutional neural network. IEEE Access, 2019. 7: p. 92871-92880.
18. Attia, Z. I., et al., Screening for cardiac contractile dysfunction using an artificial intelligence-enabled electrocardiogram. Nat Med, 2019. 25(1): p. 70-74.
19. Leong, D. P., et al., From ACE inhibitors/ARBs to ARNIs in coronary artery disease and heart Failure (Part 2/5). Journal of the American College of Cardiology, 2019. 74(5): p. 683-698.
20. Yamani, H., Q. Cai, and M. Ahmad, Three-dimensional echocardiography in evaluation of left ventricular indices. Echocardiography, 2012. 29(1): p. 66-75.
21. Quiiones, M. A., et al., A new, simplified and accurate method for determining ejection fraction with two-dimensional echocardiography. Circulation, 1981. 64(4): p. 744-753.
22. Lang, R. M., et al., Recommendations for cardiac chamber quantification by echocardiography in adults: an update from the American Society of Echocardiography and the European Association of Cardiovascular Imaging. European Heart Journal-Cardiovascular Imaging, 2015. 16(3): p. 233-271.
23. Farsalinos, K. E., et al., Head-to-head comparison of global longitudinal strain measurements among nine different vendors: the EACVI/ASE Inter-Vendor Comparison Study. Journal of the American Society of Echocardiography, 2015. 28(10): p. 1171-1181. e2.
24. Ouyang, D., et al., Video-based AI for beat-to-beat assessment of cardiac function. Nature, 2020. 580(7802): p. 252-256.
25. Attia, Z. I., et al., Screening for cardiac contractile dysfunction using an artificial intelligence-enabled electrocardiogram. Nature medicine, 2019. 25(1): p. 70-74.
26. Yao, X., et al., Artificial intelligence-enabled electrocardiograms for identification of patients with low ejection fraction: a pragmatic, randomized clinical trial. Nature Medicine, 2021: p. 1-5.
27. Spaccarotella, C. A. M., et al., Multichannel electrocardiograms obtained by a Smartwatch for the diagnosis of ST-segment changes. JAMA cardiology, 2020. 5(10): p. 1176-1180.
28. Shrikumar, A., P. Greenside, and A. Kundaje. Learning important features through propagating activation differences. in International Conference on Machine Learning. 2017. PMLR.
29. Wang, F., L. P. Casalino, and D. Khullar, Deep learning in medicine-promise, progress, and challenges. JAMA internal medicine, 2019. 179(3): p. 293-294.
30. Wang, F., R. Kaushal, and D. Khullar, Should health care demand interpretable artificial intelligence or accept “black box” medicine? 2020, American College of Physicians.
31. Rudin, C., Stop explaining black box machine learning models for high stakes decisions and use interpretable models instead. Nature Machine Intelligence, 2019. 1(5): p. 206-215.
32. Lampert, J., et al., Prognostic Value of Electrocardiographic QRS Diminution in Patients With COVID-19. Journal of the American College of Cardiology, 2021. 77(17): p. 2258-2259.
33. Babic, B., et al., Direct-to-consumer medical machine learning and artificial intelligence applications. Nature Machine Intelligence, 2021. 3(4): p. 283-287.
34. Investigators, A., Relationships between sinus rhythm, treatment, and survival in the Atrial Fibrillation Follow-Up Investigation of Rhythm Management (AFFIRM) Study. Circulation, 2004. 109(12): p. 1509-1513.
35. Abraham, W. T. and D. L. Hayes, Cardiac resynchronization therapy for heart failure. Circulation, 2003. 108(21): p. 2596-2603.
36. Chen, I. Y., et al., Ethical Machine Learning in Healthcare. Annual Review of Biomedical Data Science, 2020. 4.
37. Bellamy, R. K., et al., AI Fairness 360: An extensible toolkit for detecting and mitigating algorithmic bias. IBM Journal of Research and Development, 2019. 63(4/5): p. 4: 1-4: 15.
38. Dewland, T. A., et al., Incident atrial fibrillation among Asians, Hispanics, blacks, and whites. Circulation, 2013. 128(23): p. 2470-2477.
39. Zhang, C., et al., Understanding deep learning requires rethinking generalization. arXiv preprint arXiv:1611.03530, 2016.
40. Lachish, S. and K. A. Murray, The certainty of uncertainty: potential sources of bias and imprecision in disease ecology studies. Frontiers in veterinary science, 2018. 5: p. 90.
41. Zemel, R., et al., Learning fair representations. in International conference on machine learning. 2013. PMLR.
42. Gichoya, J. W., et al., Equity in essence: a call for operationalising fairness in machine learning for healthcare. BMJ Health & Care Informatics, 2021. 28(1): p. e100289.
43. Streltsov, A., G. Adesso, and M. B. Plenio, Colloquium: Quantum coherence as a resource. Reviews of Modern Physics, 2017. 89(4): p. 041003.
44. Popmintchev, T., et al., Bright coherent ultrahigh harmonics in the keV x-ray regime from mid-infrared femtosecond lasers. science, 2012. 336(6086): p. 1287-1291.
45. Hussein, M. I., C. N. Tsai, and H. Honarvar, Thermal conductivity reduction in a nanophononic metamaterial versus a nanophononic crystal: a review and comparative analysis. Advanced Functional Materials, 2020. 30(8): p. 1906718.
46. Honarvar, H. and M. I. Hussein, Two orders of magnitude reduction in silicon membrane thermal conductivity by resonance hybridizations. Physical Review B, 2018. 97(19): p. 195413.
47. Mittal, R., et al., Computational modeling of cardiac hemodynamics: current status and future outlook. Journal of Computational Physics, 2016. 305: p. 1065-1082.
48. Attia, Z. I., et al., An artificial intelligence-enabled ECG algorithm for the identification of patients with atrial fibrillation during sinus rhythm: a retrospective analysis of outcome prediction. The Lancet, 2019. 394(10201): p. 861-867.
49. Coumel, P., P. MAISON-BLANCHE, and D. Catuli, Heart rate and heart rate variability in normal young adults. Journal of cardiovascular electrophysiology, 1994. 5(11): p. 899-911.
50. He, K., et al., Identity mappings in deep residual networks. in European conference on computer vision. 2016. Springer.
51. Ioffe, S. and C. Szegedy. Batch normalization: Accelerating deep network training by reducing internal covariate shift. in International conference on machine learning. 2015. PMLR.
52. Srivastava, N., et al., Dropout: a simple way to prevent neural networks from overfitting. The journal of machine learning research, 2014. 15(1): p. 1929-1958.
53. He, K., et al., Delving deep into rectifiers: Surpassing human-level performance on imagenet classification, in Proceedings of the IEEE international conference on computer vision. 2015.
54. Kingma, D. P. and J. Ba. Adam: A Method for Stochastic Optimization, in ICLR (Poster). 2015.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A computer system for determining whether a subject has a cardiovascular abnormality, the computer system comprising:
one or more processors; and
memory addressable by the one or more processors, the memory storing at least one program for execution by the one or more processors, the at least one program comprising instructions for:
(A) obtaining an electrocardiogram of the subject, wherein the electrocardiogram represents a total time interval, the electrocardiogram comprising electronic measurements for a plurality of leads;
(B) obtaining, for each lead in the plurality of leads, a corresponding plurality of sub-waveforms from the electrocardiogram, the plurality of sub-waveforms having a common duration that is less than the total time interval, wherein the common duration represents a fixed multiple of a reference heartbeat duration, and wherein each of the sub-waveforms in the plurality of sub-waveform being offset from a beginning of the electrocardiogram by a unique respective multiple of a sliding parameter; and
(C) inputting the corresponding plurality of sub-waveforms for each lead in the plurality of leads into a neural network, thereby obtaining an indication as to whether the subject has the cardiovascular abnormality.

2. The computer system of claim 1, wherein the cardiovascular abnormality is left ventricular systolic dysfunction (LVSD) or asymptomatic left ventricular dysfunction (ALVD).

3. The computer system of claim 1, wherein the cardiovascular abnormality is right ventricular dysfunction.

4-6. (canceled)

7. The computer system of claim 1, wherein the total time interval is X seconds, wherein X is a positive integer of 5 or greater, the reference heartbeat duration is between 0.70 seconds and 0.78 seconds, and the fixed multiple is a scalar value between 2 and 30.

8. (canceled)

9. The computer system of claim 1, wherein the indication is in a binary discrete classification form in which a first possible value outputted by the neural network indicates the subject has the cardiovascular abnormality; and a second possible value outputted by the neural network indicates the subject does not have the cardiovascular abnormality.

10. The computer system of claim 25, wherein the indication is in a scalar form indicating a likelihood or probability that the subject has the cardiovascular abnormality or a likelihood or probability that the subject does not have the cardiovascular abnormality.

11. The computer system of claim 3, wherein the neural network is a convolutional neural network having a plurality of convolutional layers followed by a plurality of residual blocks, wherein each residual block in the plurality of residual blocks comprises a pair of convolutional layers, followed by a fully connected layer followed by a sigmoid function.

12. The computer system of claim 1, wherein the electronic measurements for each of the plurality of leads comprise a plurality of data points representing electronic measurements at different respective time intervals of a plurality of time intervals summing to the total time interval, the method further comprising standardizing the plurality of data points of each respective lead in the plurality of leads to have zero-mean and unit-variance.

13. The computer system of claim 1, wherein the neural network comprises a plurality of parameters, the method further comprising:
(D) using the plurality of parameters to compute a saliency map of at least a subset of the plurality of parameters.

14. The computer system of claim 1, wherein the electronic measurements for each of the plurality of leads comprise a plurality of data points representing electronic measurements at different respective time intervals of a plurality of time intervals summing to the total time interval, the method further comprising:
(D) using an occlusion approach to determine a weighting of effect on the indication for each data point in the plurality of data points for at least one lead in the plurality of leads.

15. The computer system of claim 14, wherein the occlusion approach is forward pass attribution.

16. (canceled)

17. A method for determining whether a subject has a cardiovascular abnormality, the method comprising:
at a computer system comprising a memory:
(A) obtaining an electrocardiogram of the subject, wherein the electrocardiogram represents a total time interval, the electrocardiogram comprising electronic measurements; for a plurality of leads;
(B) obtaining, for each lead in the plurality of leads, a corresponding plurality of sub-waveforms from the electrocardiogram, the plurality of sub-waveforms having a common duration that is less than the total time interval, wherein the common duration represents a fixed multiple of a reference heartbeat duration, and wherein each of the sub-waveforms in the plurality of sub-waveform is offset from a beginning of the electrocardiogram by a unique respective multiple of a sliding parameter; and
(C) inputting the corresponding plurality of sub-waveforms for each lead in the plurality of leads into a neural network, thereby obtaining an indication as to whether the subject has the cardiovascular abnormality.

18. A non-transitory computer readable storage medium, wherein the non-transitory computer readable storage medium stores instructions, which when executed by a computer system, cause the computer system to perform a method for determining whether a subject has a cardiovascular abnormality, the method comprising:
(A) obtaining an electrocardiogram of the subject, wherein the electrocardiogram represents a total time interval, the electrocardiogram comprising electronic measurements for a plurality of leads;
(B) obtaining, for each lead in the plurality of leads, a corresponding plurality of sub-waveforms from the electrocardiogram, the plurality of sub-waveforms having a common duration that is less than the total time interval, wherein the common duration represents a fixed multiple of a reference heartbeat duration, and wherein each of the sub-waveforms in the plurality of sub-waveform is offset from a beginning of the electrocardiogram by a unique respective multiple of a sliding parameter; and
(C) inputting the corresponding plurality of sub-waveforms for each lead in the plurality of leads into a neural network thereby obtaining an indication as to whether the subject has the cardiovascular abnormality.

19. The computer system of claim 1, wherein the common duration represents at least two reference heartbeat durations.

20. The computer system of claim 1, wherein the sliding parameter represents a multiple of a reference heartbeat duration.

21. The computer system of claim 1, wherein the sliding parameter is a no greater than the common duration.

22. The computer system of claim 21, the sliding parameter is smaller than the common duration.

23. The computer system of claim 22, wherein the common duration is two reference heartbeat durations and the sliding parameter is one reference heartbeat duration.

24. The computer system of claim 22, wherein the common duration is four reference heartbeat durations and the sliding parameter is two reference heartbeat durations.

25. The computer system of claim 1, wherein the common duration represents an optimal fixed multiple of the reference heartbeat, optimal fixed multiple being determined, among multiple values of the fixed multiple, based on a measured prediction performance.
